(19) United States
(12) Patent Application Publication
(10) Pub. No.: US 2007/0238647 A1
(43) Pub. Date: Oct. 11, 2007

(54) Methods for treating prostate cancer

(75) Inventors: Richard Lloyd Bowen, Raleigh, NC (US); Christopher W. Gregory, Cary, NC (US)

Correspondence Address: COVINGTON & BURLING, LLP;ATTN: PATENT DOCKETING, 1201 PENNSYLVANIA AVENUE, N.W., WASHINGTON, DC, 20004-2401, US

(73) Assignee: Voyager Pharmaceutical Corporation, Raleigh, NC (US)

(21) Appl. No.: 11/808,801

(22) Filed: Jun. 13, 2007

Related U.S. Application Data
(63) Continuation of application No. 11/180,667, filed on Jul. 14, 2005, now abandoned.

(51) Int. Cl. A61K 48/00

(52) U.S. Cl. 514/2

(57) ABSTRACT

Methods are provided for treating prostate cancer, preventing or slowing proliferation of cells of prostate origin, preventing prostate cancer in a patient at risk of contracting prostate cancer, preventing or inhibiting an upregulation of the cell cycle in prostate-derived cells in a patient, and decreasing the level of prostate-specific antigen in a patient.

### RELATED APPLICATIONS

This application is a continuation of U.S. application Ser. No. 11/180,667, filed Jul. 14, 2005, the entire teaching of which is incorporated herein by reference.

### FIELD OF THE INVENTION

The present invention relates to treating, mitigating, slowing the progression of, or preventing prostate cancer or preventing or slowing proliferation of cells of prostate origin.

### BACKGROUND

Prostate cancer is the most common cancer in American men, with more than 230,000 new cases diagnosed each year. Approximately 30,000 deaths will be attributed to prostate cancer in 2004 (Jemal A, Tiwari R C, Murray T. Ghafoor A, Samuels A, Ward E, Feuer E J, Thun M J. Cancer statistics 2004. CA Cancer J. Clin. 54:8-29, 2004).

When initially diagnosed, prostate cancer in most patients is managed with either: close observation without any intervention, termed “watchful waiting”: or surgical removal of the prostate, termed “radical prostatectomy”: or with radiation by placing radioactive pellets into the prostate, termed “brachytherapy.” Radical prostatectomy and brachytherapy are frequently preceded by short-term hormonal treatment. In addition, depending on the patient's condition and preferences, other therapies may be used.

Approximately 40% of individuals treated with surgery or radiation will develop recurrent prostate cancer (Walsh P C, Retik A B, Vaughan E D, eds. Campbell's Urology. 7th ed. Philadelphia, Pa.: W B Saunders Company; 1998). The most common treatment for recurrent prostate cancer is the suppression of testicular testosterone production via orchiectomy, estrogen treatment, antiandrogen administration, and/or GnRH agonist/antagonist treatment. This usually results in remission for 2-3 years, after which time prostate cancer becomes “hormone refractory,” meaning that it develops the ability to grow despite the reduction of blood androgen concentrations to castrate levels.

For patients initially diagnosed with metastatic disease, it is already too late to perform brachytherapy or a radical prostatectomy. Therefore, these patients are typically treated initially with some type of testosterone suppressive therapy. Once their cancer becomes hormone refractory, the median survival is 10-24 months.

In order to provide an understanding of hormonal therapy, a brief overview of the hypothalamic-pituitary-gonadal (HPG) hormonal axis is presented with reference to FIG. 11. Activins, which are produced by most tissues, stimulate gonadotropin releasing hormone (GnRH) secretion from the hypothalamus which stimulates the anterior pituitary to secrete the gonadotropins, LH and FSH, which in turn enter the bloodstream and bind to receptors in the gonads and stimulate oogenesis/spermatogenesis as well as sex steroid and inhibin production. (Reichlin S. Neuroendocrinology; in Wilson J D, Foster D W, Kronenberg H M, Larsen P R 9eds): William's Textbook of Endocrinology, ed. 9. Philadelphia, Saunders, 1998, pp. 165-248). The sex steroids and inhibin then feed back to the hypothalamus and pituitary, resulting in a decrease in gonadotropin secretion. (Thorner M, Vance M, Laws E Jr., Horvath E, Kovacs K. The anterior pituitary; in Wilson J D, Foster D W, Kronenberg H M, Larsen P R 9eds): William's Textbook of Endocrinology, ed. 9. Philadelphia, Saunders, 1998, pp. 249-340).

GnRH agonists are the most commonly used type of hormonal therapy. These are analogues of the endogenous GnRH decapeptide with specific amino acid substitutions. Replacement of the GnRH carboxy-terminal glycinamide residue with an ethylamide group greatly increases the affinity these analogues possess for the GnRH receptor compared to the endogenous peptide. Many of these analogues also have a longer half-life than endogenous GnRH (Millar R P, Lu Z L, Pawson A J, Flanagan C A, Morgan K, Maudsley S R. Gonadotropin-releasing hormone receptors. Endocrine Reviews 25:235-275, 2004). Administration results in an initial increase in serum gonadotropin concentrations that persists for several days (there is also a corresponding increase in testosterone in men and estrogen in pre-menopausal women). This is followed by a precipitous decrease in gonadotropins. This suppression is due to the loss of GnRH signaling due to down regulation of pituitary GnRH receptors (Belchetz P E, Plant T M, Nakai Y, Keogh E J, Knobil E. Hypophysial responses to continuous and intermittent delivery of hypothalamic gonadotropin-releasing hormone. Science 202:631-633, 1978). This is thought to be secondary to the increased concentration of ligand, the increased affinity of the ligand for the receptor, and the continuous receptor exposure to ligand as opposed to the intermittent exposure that occurs with physiological pulsatile secretion.

The underlying rationale for using hormonal therapy in the treatment of prostate cancer is the suppression of androgens in the bloodstream to concentrations seen with castration. Therefore, once this was achieved there was no reason to continue to escalate doses of such therapies. However, the present invention provides that higher doses, meaning doses that achieve and maintain higher serum or tissue concentrations of GnRH agonists or antagonists, are more effective at treating, mitigating, slowing the progression of, or preventing prostate cancer.

Leuprolide acetate is an example of a GnRH agonist used in the treatment of prostate cancer. Approved GnRH agonists and antagonists, dosage levels and plasma/serum levels of active medication are as follows (according to their approved labelling): LUPRON® DEPOT 3.75 mg 1 month injection gives a mean plasma leuprolide concentration of 4.6-10.2 ng/ml at 4 hours postdosing; LUPRON® DEPOT 7.5 mg 1 month injection gives a mean plasma leuprolide concentration of 20 ng/ml at 4 hours and 0.36 ng/ml at 4 weeks; LUPRON® DEPOT-PED 11.25 mg 1 month injection gives a mean plasma leuprolide concentration of 1.25 ng/ml at 4 weeks; LUPRON® DEPOT-PED 15mg injection gives a mean plasma leuprolide concentration of 1.59 ng/ml at 4 weeks; LUPRON® DEPOT 22.5 mg 3 month injection gives a mean plasma leuprolide concentration of 48.9 ng/ml at 4 hours and 0.67 ng/ml at 12 weeks; LUPRON® DEPOT 30 mg 4 month injection gives a mean plasma leuprolide concentration of 59.3 ng/ml at 4 hours and 0.3 ng/ml at 16 weeks; VIADUR® 72 mg 12 month implantation gives a mean serum leuprolide concentration of 16.9 ng/ml at 4 hours and 2.4 ng/ml at 24 hours with a 0.9 ng/ml mean serum concentration for 12 months; ELIGARD® 7.5 mg 1 month injection gives a mean serum leuprolide concentration of 25.3 ng/ml at 5 hours and a serum level range of 0.28-2.0 ng/ml for one month; ZOLADEX® 3.6mg 1 month (serum levels unavailable); ZOLADEX® 10.8 mg 3 month (serum levels unavailable); SYNAREL® 200 micrograms twice daily (serum levels unavailable); TRELSTAR DEPOT 3.75 mg 1 month gives a mean plasma triptorelin concentration of 28.43 ng/ml at 4 hours and declines to 0.084 ng/ml at 4 weeks; Supprelin 200 μg/ml, 500 μg/ml and 1000 μg/ml for daily injection (serum levels unavailable); SUPREFACT® 6.3 mg 2 month implant or 500 μg every 8 hours for 7 days followed by 200 μg per day (serum levels unavailable); CETROTIDE® 0.25 mg daily or 3.0 mg every 4 days gives a mean plasma cetrorelix concentration of 4.97 ng/ml or 28.5 ng/ml at 4 hours, respectively; PLENAXIS® 100 mg given on days 1, 15 and 28 and every 4 weeks afterward (serum levels unavailable); ANTAGON 250 μg daily gives a mean plasma ganirelix concentration of 14.8 ng/ml at 4 hours.

Typically, when leuprolide acetate is administered in a particular depot form, most of the drug (up to 80% to 90% of the total amount available in the depot) is released in the first few days. Then, the remainder is released over the next several weeks. In the case of a 22.5 mg-3 month injection, the large majority of the drug may be released into the bloodstream within the first week, with the remaining fraction released over the next eleven or so weeks. Thus, while there is an initial spike in serum concentration of leuprolide acetate—up to 15 or 20 ng/ml, for example—thereafter the serum concentration drops markedly and remains much lower for the rest of the 3-month period.

### SUMMARY

Since prostate cancer has traditionally been thought to be driven by testicular androgens, and maximal inhibition of testicular androgen production can be reached with current doses of GnRH agonists and antagonists, then under the conventional teaching there is no reason to escalate doses of such drugs in the treatment of prostate cancer. However, preclinical prostate cancer data provided herein indicate that higher concentrations of GnRH agonists are more effective at inhibiting growth of cell lines and tumors. The present invention provides, therefore, that suppression of the autocrine/paracrine GnRH signaling in the prostate requires doses of GnRH agonists that are significantly higher than those required to suppress endocrine GnRH signaling at the level of the pituitary.

Normal as well as cancerous prostate tissue expresses hormones and their respective cognate receptors involved in the HPG axis, including: activins, inhibins, follistatin, gonadotropin releasing hormone (GnRH), follicle stimulating hormone (FSH), luteinizing hormone (LH), and sex steroids. The present invention provides that hormones of the hypothalamic-pituitary-gonadal (HPG) axis function not only in an endocrine fashion to modulate prostate cell function but also in an autocrine/paracrine fashion to regulate prostate cell function. While customary doses of GnRH agonists and antagonists are generally considered to be adequate to suppress the endocrine influences of testosterone and possibly other hormones by significantly lowering their serum concentrations (produced by hypothalamus, pituitary, and testicles), these same doses of GnRH antagonists and agonists are believed to be subtherapeutic when it comes to treating, mitigating, slowing the progression of, or preventing prostate cancer.

Among the goals of the present invention is treatment, mitigation, slowing the progression of, or preventing prostate cancer by achieving higher tissue levels of GnRH agonists and/or GnRH antagonists, whether by administering more of such drugs, by preventing degradation of such drugs once administered, by delivering the drugs at a site where they are needed, by a combination of these methods, or by other methods.

The present invention relates to methods for treating, mitigating, slowing the progression of, or preventing prostate cancer, or preventing or slowing proliferation of cells of prostate origin, or for decreasing the level of prostate-specific antigen in a patient, by administering high doses of at least one physiological agent, such as a GnRH agonist or a GnRH antagonist, that decreases or regulates the blood or tissue levels, expression, production, function, or activity of LH, LH receptors, FSH, FSH receptors, androgenic steroids, androgenic steroid receptors, activins, or activin receptors, or administering a physiological agent that increases or regulates the blood or tissue levels, expression, production, function, or activity of GnRH, inhibins, beta-glycan, or follistatins.

The invention further encompasses, for example, a method of preventing or inhibiting an upregulation of the cell cycle in prostate-derived cells by administering high doses of at least one physiological agent that is a GnRH agonist or antagonist, effective to reduce local tissue production of hormones of the hypothalamic-pituitary-gonadal (HPG) axis. In embodiments, the physiological agent is leuprolide, and the amount administered is in the range of approximately at least 15 mg/month. In other embodiments, the amount of leuprolide administered is in the range of at least about 20 mg/month, or at least 37.5 mg/month. In other embodiments, the physiological agent is an agent other than leuprolide, and the amount administered is an amount sufficient to produce the same or similar physiological effects as at least about 15 mg of leuprolide per month, or at least about 20 mg of leuprolide per month, or at least about 37.5 mg of leuprolide per month. In this specification, the term “physiologically equivalent dose” to a dose of a first physiological agent means a dose of a second physiological agent that achieves the same or similar physiological responses as the dose of the first physiological agent. The present invention further encompasses, as a further example, a method for treating prostate cancer comprising administering to the patient an amount of at least one physiological agent selected from the group consisting of GnRH agonists and GnRH antagonists, effective to achieve a blood serum level of at least 3 ng/ml of the physiological agent for a predetermined period of time, such as at least one month or at least three months. Similarly, the present invention encompasses a method of treating prostate cancer by administering a physiological agent in an amount, administered or released over a predetermined time period (e.g., at least one month or at least three months), targeted to achieve substantially equivalent physiological effects as those resulting from a blood serum level of leuprolide of at least about 3 ng/ml over about the predetermined time period.

As another example, the present invention also encompasses a method for treating prostate cancer comprising administering to a patient an initial dose of a GNRH agonist or a GnRH antagonist, monitoring for decreases in prostate-specific antigen level in the patient, and subsequently administering to the patient increasing doses of the GnRH agonist or the GNRH antagonist until no further decrease in prostatic-specific antigen level is observed.

### BRIEF DESCRIPTION OF THE DRAWINGS

FIG. 1A presents results of an in vitro experiment in which leuprolide acetate was administered to cells of the DU 145 recurrent (androgen-insensitive) prostate cancer line on the initial day of a seven-day period.

FIG. 1B presents results of an in vitro experiment in which leuprolide acetate was administered to cells of the DU 145 recurrent (androgen-insensitive) prostate cancer line on the initial and third days of a seven-day period.

FIG. 1C presents results of an in vitro experiment in which leuprolide acetate was administered to cells of the DU 145 recurrent (androgen-insensitive) prostate cancer line on each day of a seven-day period.

FIG. 2A presents results of an in vitro experiment in which leuprolide acetate was administered to cells of the PC3 recurrent (androgen-insensitive) prostate cancer line on the initial day of a seven-day period.

FIG. 2B presents results of an in vitro experiment in which leuprolide acetate was administered to cells of the PC3 recurrent (androgen-insensitive) prostate cancer line on the initial and third days of a seven-day period.

FIG. 2C presents results of an in vitro experiment in which leuprolide acetate was administered to cells of the PC3 recurrent (androgen-insensitive) prostate cancer line on each day of a seven-day period.

FIG. 3A presents results of an in vitro experiment in which leuprolide acetate was administered to cells of the CWR-R1 recurrent (androgen-sensitive) prostate cancer line on the initial day of a seven-day period.

FIG. 3B presents results of an in vitro experiment in which leuprolide acetate was administered to cells of the CWR-R1 recurrent (androgen-sensitive) prostate cancer line on the initial and third days of a seven-day period.

FIG. 3C presents results of an in vitro experiment in which leuprolide acetate was administered to cells of the CWR-R1 recurrent (androgen-sensitive) prostate cancer line on each day of a seven-day period.

FIG. 3D presents results of an in vitro experiment in which leuprolide acetate was administered to cells of the CWR-RI recurrent (androgen-sensitive) prostate cancer cell line twice a day on each day of a five-day period.

FIG. 4A presents results of an in vitro experiment in which leuprolide acetate was administered to cells of the LNCaP androgen-sensitive prostate cancer line on the initial day of a seven-day period.

FIGS. 4B and 4C present results of an in vitro experiment in which leuprolide acetate was administered to cells of the LNCaP androgen-sensitive prostate cancer line on each day of a seven-day period. In results presented in FIG. 4B, 20,000 cells were plated initially. In results presented in FIG. 4C, 100,000 cells were plated initially.

FIG. 4D presents results of an in vitro experiment in which leuprolide acetate was administered to cells of the LNCaP androgen-sensitive prostate cancer line twice daily over a five-day period.

FIG. 5A presents tumor growth data from an experiment in which human LNCaP prostatic cancer cells were injected as xenografts into nude mice that one week before had been treated with placebo or leuprolide implants.

FIG. 5B presents tumor growth rate results from the same experiment represented in FIG. 5A.

FIG. 6A presents tumor growth data for small tumors from an experiment in which human DU 145 prostatic cancer cells were injected as xenografts into nude mice with concurrent implantation of placebo or leuprolide implants.

FIG. 6B presents tumor growth rate results for small tumors from the same experiment represented in FIG. 6A.

FIG. 6C presents tumor growth data for large tumors from the same experiment represented in FIG. 6A.

FIG. 6D presents tumor growth rate for large tumors from the same experiment represented in FIG. 6A.

FIG. 7A presents tumor growth data from a replicate experiment in which human DU 145 prostatic cancer cells were injected as xenografts into nude mice with implantation one week before of placebo or leuprolide implants.

FIG. 7B presents tumor growth rate from the same experiment represented in FIG. 7A.

FIG. 8 presents tumor growth data from an experiment in which human DU 145 prostatic cancer cells were injected as xenografts into nude mice and allowed to establish for one week prior to implantation of placebo or leuprolide implants.

FIG. 9A presents tumor growth data from an experiment in which human CWR22 recurrent prostate cancer cells were injected as xenografts into nude mice with concurrent implantation of placebo or leuprolide implants.

FIG. 9B presents tumor growth rate results from the same experiment represented in FIG. 9A.

FIG. 9C presents tumor growth data from a replicate experiment in which human CWR22 recurrent prostate cancer cells were injected as xenografts into nude mice with concurrent implantation of placebo or leuprolide implants.

FIG. 9D presents tumor growth rate results from the same experiment represented in FIG. 9C.

FIG. 10A presents tumor growth data from an experiment in which human CWR22 recurrent prostate cancer cells were injected as xenografts into nude mice with implantation four days before of placebo or leuprolide implants.

FIG. 10B presents tumor growth rate results from the same experiment represented in FIG. 10A.

FIG. 10C presents tumor growth data from a replicate experiment in which human CWR22 recurrent prostate cancer cells were injected as xenografts into nude mice with implantation three days before of placebo or leuprolide implants.

FIG. 10D presents tumor growth rate results from the same experiment represented in FIG. 10C.

FIG. 11 is a schematic overview of the hypothalamic-pituitary-gonadal hormonal axis.

### DETAILED DESCRIPTION

Among the methods provided by the present invention are methods of treating, mitigating or slowing the progress of prostate cancer, preventing or slowing proliferation of cells of prostate origin, preventing prostate cancer in a patient at risk of contracting prostate cancer, or decreasing the level of prostate-specific antigen in a patient, in which therapeutically effective amounts of at least one physiological agent, or therapeutically effective combinations of physiological agents, are administered to a patient. “Therapeutically effective” in these instances means that the amount or the combination is effective to reduce or suppress local tissue production of hormones of the hypothalamic-pituitary-gonadal (HPG) axis. For example, a therapeutically effective amount of a GnRH agonist as used in the present invention is expected. to be higher than the current doses used in the treatment, prevention, mitigation, or slowing the progress of prostate cancer.

The present invention is expected to be useful in treating all prostate cancer, but in particular, it is believed that the invention can be useful in treating, mitigating, slowing the progress of, and preventing the hormone refractory prostate cancer which occurs after androgen deprivation therapy has failed and in which the disease continues to progress in the presence of castrate serum levels of androgen in the bloodstream.

GnRH Agonists and Antagonists

The mainstays of current androgen deprivation therapy are the GnRH agonists. GnRH agonists were developed as a method of suppressing sex steroid production as an alternative to surgical castration in the treatment of advanced prostate cancer. GnRH agonists are analogues of the endogenous GnRH decapeptide with specific amino acid substitutions. Replacement of the GnRH carboxyl-terminal glycinamide residue with an ethylamide group greatly increases the affinity of these analogues for the GnRH receptor compared to the endogenous peptide. Many of these analogues also have a longer half-life than endogenous GNRH. Administration results in an initial increase in serum gonadotropin concentrations that persists for several days (there is also a corresponding increase in testosterone in men and in estrogen in pre-menopausal women). This is followed by a precipitous decrease in gonadotropins and sex steroids. This suppression is thought to be secondary to the loss of GnRH signaling due to down-regulation of pituitary GnRH receptors (Belchetz, P. E., Plant, T. M., Nakai, Y., Keogh, E. J., and Knobil, E. (1978) Hypophysial responses to continuous and intermittent delivery of hypothalamic gonadotropin-releasing hormone. Science 202:631-633). This is a likely consequence of the increased concentration of ligand, the increased affinity of the ligand for the GnRH receptor, and the continuous receptor exposure to ligand, as opposed to the intermittent exposure that occurs with physiological pulsatile secretion. By this mechanism, chronic administration of GnRH agonists inhibits testicular steroidogenesis, thereby reducing the levels of circulating androgens to castrate levels (≦50 ng/dL). This results in reversible medical castration, a mainstay therapeutic strategy for advanced, metastatic prostate cancer.

GnRH antagonists have also been developed for use in the treatment of prostate cancer. The GnRH antagonists were developed to inhibit gonadotropin and sex steroid synthesis and secretion without the initial spike in gonadotropins and sex steroids associated with GnRH agonists. While GnRH antagonists do prevent this initial burst, there is more “breakthrough” in LH and testosterone secretion than with GNRH agonists (Praecis Pharmaceuticals Incorporated, Plenaxis Package Insert. 2004). This may be due to a compensatory increase in hypothalamic GnRH secretion which alters the ratio of the competing ligands, resulting in activation of the receptor. In contrast, with GnRH agonists, a compensatory increase in hypothalamic GnRH would serve to potentiate receptor down-regulation. In addition to this efficacy issue, GnRH antagonists are associated with occasional anaphylactic reactions due to their high histamine releasing properties (Millar, R. P., Lu, Z. L., Pawson, A. J., Flanagan, C. A., Morgan, K., and Maudsley, S. R. (2004) Gonadotropin-releasing hormone receptors. Endocr. Rev. 25:235-275). Therefore, for chronic use, the GnRH agonists are often preferred as more effective than the GnRH antagonists at suppressing gonadotropins.

Since these GnRH agonists are peptides, they are generally not amenable to oral administration. Therefore, they are usually administered subcutaneously, intramuscularly, or via nasal spray. GnRH agonists are highly potent with serum concentrations of less than 1 ng/ml of leuprolide acetate required for testosterone suppression (Fowler, J. E., Flanagan, M., Gleason, D. M., Klimberg, I. W., Gottesman, J. E., and Sharifi, R. (2000) Evaluation of an implant that delivers leuprolide for 1 year for the palliative treatment of prostate cancer. Urol. 55:639-642). Due to their small size and high potency, GnRH agonists are also often considered to be ideal for use in long-acting depot delivery systems. At least ten such products are currently marketed in the United States. The duration of action of these products ranges from one month to one year. Leuprolide acetate has been on the market for close to two decades and continues to demonstrate a favorable side effect profile. Most of the side effects such as hot flashes and osteoporosis can be attributed to the loss of sex steroid production (Stege, R. (2000). Potential side-effects of endocrine treatment of long duration in prostate cancer. Prostate Suppl. 10:38-42). Leuprolide acetate is currently available, for example, in a 7.5 mg single dose, administered as a monthly injection (LUPRON DEPOT® 7.5 mg), and in other formulations identified above.

Experimental Design

The following experimental design was used in the experiments whose results are presented below. Cell growth assays were performed using two different methodologies as described below. DU145 cells were prepared by plating in Minimum essential medium (Eagle) with 2 mM L-glutamine and Earle's Balanced Salt Solution adjusted to contain 1.5 g/L sodium bicarbonate, 0.1 mM non-essential amino acids, and 1.0 mM sodium pyruvate, 90%; fetal bovine serum, 10%. PC3 cells were prepared by plating in Ham's F12K medium with 2 mM L-glutamine adjusted to contain 1.5 g/L sodium bicarbonate, 90%; fetal bovine serum, 10%. CWR-R1 cells were prepared by plating in Richter's minimum essential medium with linoleic acid (0.9 μg/ml), nicotinamide (10 mM), 20 ng/ml epidermal growth factor, 5 μg/ml selenium, 5 μg/ml insulin, 2% fetal bovine serum. LNCaP cells were prepared by plating in RPMI 1640 medium with 2 mM L-glutamine adjusted to contain 1.5 g/L sodium bicarbonate, 4.5 g/L glucose, 10 mM HEPES, and 1.0 mM sodium pyruvate, 90%; fetal bovine serum, 10%. For cell growth assays performed in a 96-well format, 5000 cells were plated per well and allowed to grow for 2 days prior to commencement of treatment. For assays performed in 60 mm×15 mm dishes, different numbers of cells were plated, depending on the cell line (2×10⁴ for DU145 and PC3, and 5×10⁴ for LNCaP and CWR-R1). After the cells were established, they were then counted in order to obtain a baseline before the various concentrations of leuprolide were administered. A 10 mM (12.25 mg/ml) solution of leuprolide acetate salt in phosphate buffered saline was prepared and diluted appropriately to obtain the desired final concentrations. Treatment concentrations were 0 M (control), 10⁻¹¹ M (shown as 1.00E-11 (0.012 μg/ml)), 10⁻⁹M (shown as 1.00E-9, (0.0012 μg/ml)), 10⁻⁸ M (shown as 1.00E-8, (0.012 μg/ml)), 10⁻⁷ M (shown) as 1.00E-7, (0.12 μg/ml)) and 10⁻⁵ M (shown as 1.00E-5, (12.25 μg/ml)). For 96-well format assays, the number of cells in each group was measured by incubating cells with WST-8 (2-(2-methoxy-4-nitrophenyl)-3-(4-nitrophenyl)-5-(2,4-disuifophenyl)-2H-tetrazolium, monosodium salt) which produces a water soluble formazan dye that was detected by measuring optical density (at 450 nm) using a μQuant™ Universal Microplate Spectrophotometer (Bio-Tek® Instruments, Inc., Winooski, Vt.). For the 60 mm dish assays, cells were counted by a blinded observer using a hemacytometer and a microscope. All treatment groups were performed in triplicate and the optical densities or cell numbers are presented as mean±standard deviation.

For prostate cancer tumor xenograft studies, male nude:nude athymic mice from Harlan Sprague Dawley (Indianapolis, Ind.) were used. Mice were anesthetized with Domitor/Ketaset and placed under a warming lamp. Tumor cells were injected in Matrigel (BD Biosciences, Bedford, Md.) and implants were placed subcutaneously into anesthetized mice. Tumor measurements were carried out twice weekly using calipers and length (1) and width (w) were converted to tumor volumes using the following equation: (w×1)/2. All tumors within one treatment group were used to calculate average tumor volumes±standard deviations. To calculate tumor growth rates, tumor volumes were normalized to the initial tumor volume (V₀). When a single tumor was detectable in a treatment group, that tumor volume was used as V₀ for that treatment group and all tumors measured in that group that formed over time were used to calculate a growth rate (V/V₀). At the end of the experiments, mice were sacrificed by cervical dislocation and tissues and blood were collected.

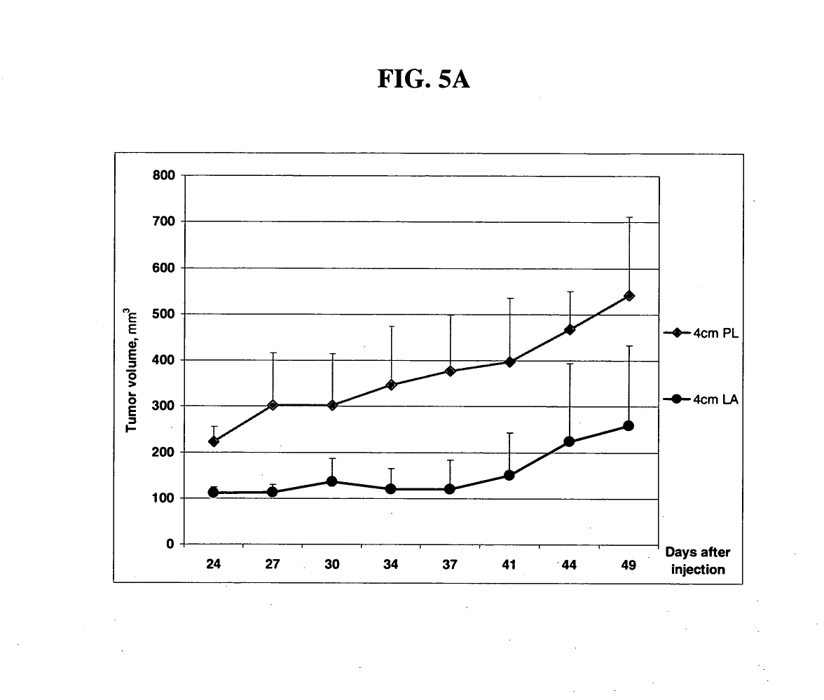

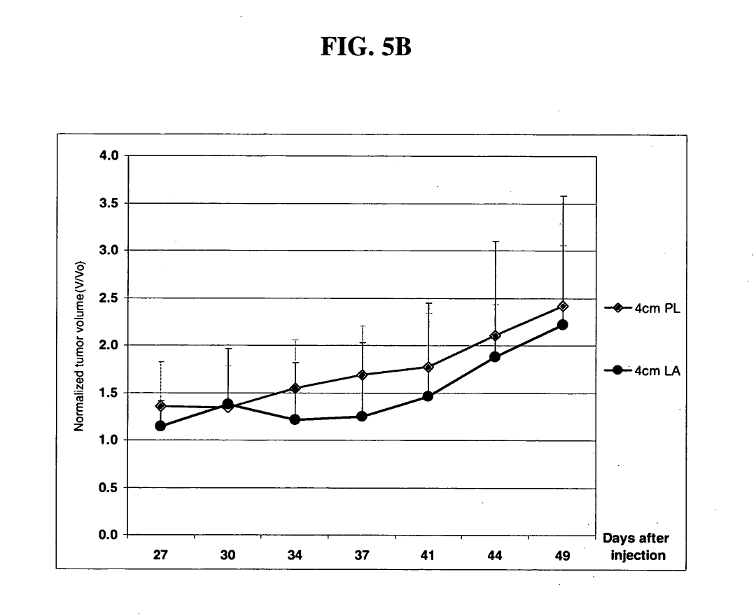

The DURIN-Leuprolide implant used in the experiments is a 2-month implant, available from Durect Corporation (Cupertino, Calif.). It is a solid formulation comprising approximately 25-30 weight % leuprolide acetate dispersed in a matrix of poly (DL-lactide-co-glycolide). The implant is a cylindrical, opaque rod with nominal dimensions of 1.5 mm (diameter)×2.0 cm (length).The formulation provides 11.25 mg of leuprolide acetate per 2 cm rod, with a substantially uniform release profile. For tumor xenograft studies, the following doses were used: placebo (2 cm of formulation, 0 mg leuprolide acetate); low dose (2 cm of formulation, 11.25 mg leuprolide acetate); medium dose (3 cm of formulation, 16.875 mg leuprolide acetate); high dose (4 cm of formulation, 22.5 mg leuprolide acetate). Accordingly, in FIGS. 5A through 10D, the dimension on the right-hand axis refers to the length of these implants used for the particular experimental group, and the designations “LA” and “PL” refer respectively to leuprolide acetate and placebo.

Experiment 1

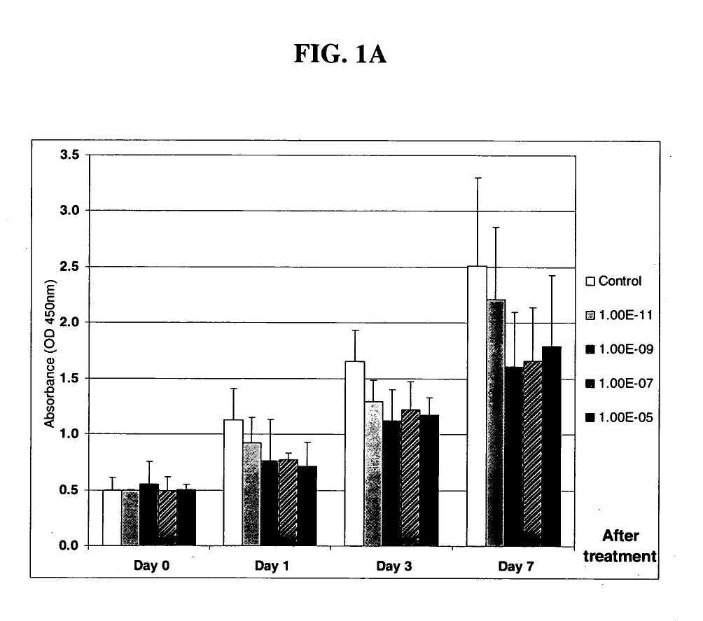

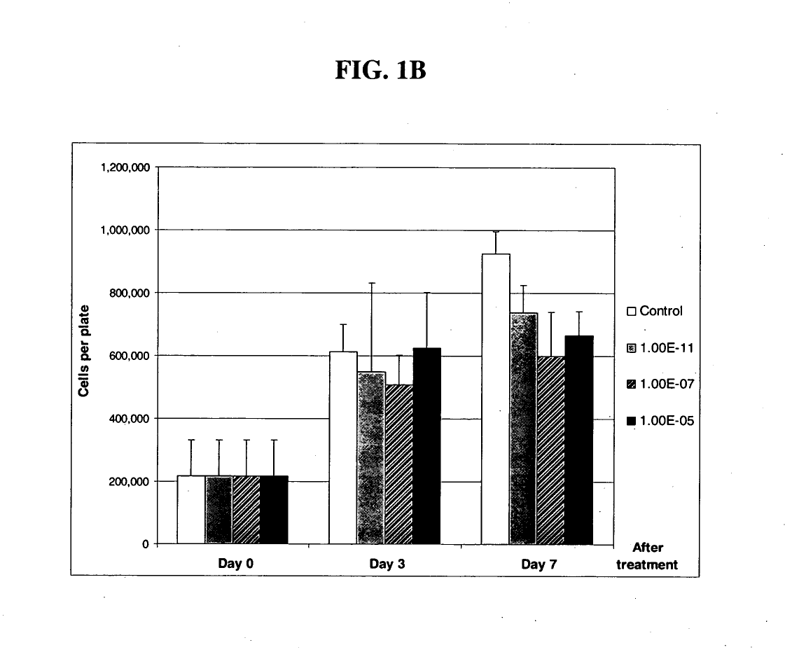

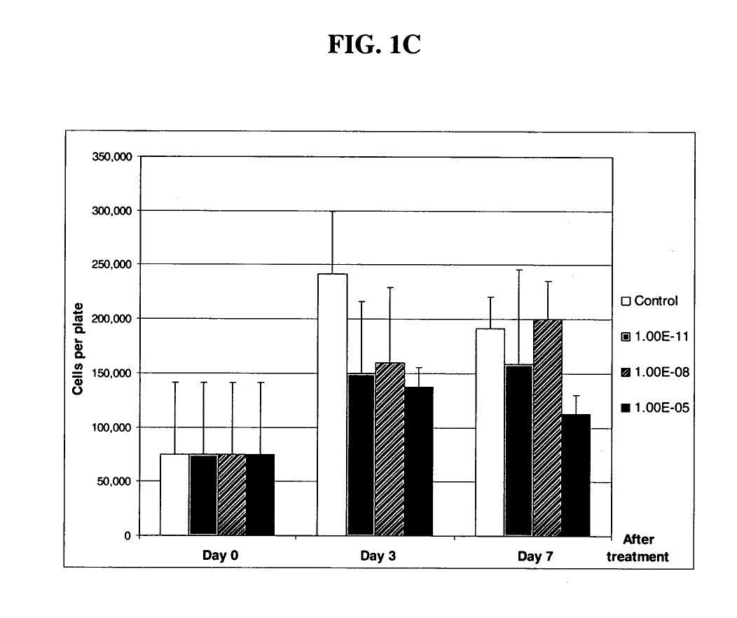

FIGS. 1A-C present results of a series of three experiments on the effects of administration of leuprolide acetate at various molar concentrations on the growth in number of cells of the DU 145 recurrent prostate cancer cell line (ATCC HTB-81, obtained from ATCC, Reston, Va.). The source of these cells was a brain lesion of a man with metastatic prostate cancer. Their characteristics include androgen insensitivity for growth and absence of expression of the androgen receptor.

In Experiment 1-A, each of four groups of cells from the DU 145 cell line was prepared as described above and respectively treated with final concentrations of 0 M (phosphate buffered saline control), 10⁻¹¹M, 10⁻⁷M or 10⁻⁵M leuprolide acetate solution at experiment commencement. The number of cells in each group was measured by incubating cells with WST-8 (as described in Experimental Design, above) at day 0 (experiment commencement) and on the first, third and seventh days following commencement. FIG. 1A presents the results of this experiment. For each concentration of leuprolide acetate used in this experiment, and for each day on which absorbance was measured, FIG. 1A shows, on the vertical axis, the absorbance (450 nm), which indicates cell number as a function of optical density of the formazan dye product.

In Experiment 1-B, each of four groups of cells from the DU 145 cell line was prepared as described above and respectively treated with final concentrations of 0 M (culture medium control), 10⁻¹¹M , 10⁻⁷M or 10⁻⁵M leuprolide acetate solution at experiment commencement and on the third day after commencement. The number of cells was measured by counting at experiment commencement and on the fourth and seventh days following commencement, using a hemacytometer and microscope by a blinded observer. FIG. 1B presents the results of this experiment. For each concentration of leuprolide acetate used in this experiment, and for each day on which cells were counted, FIG. 1B shows, on the vertical axis, the number of cells per plate.

In Experiment 1-C (DU 145), each of four groups of cells was prepared as described above and respectively treated with final concentrations of 0 M (culture medium control), 10⁻¹¹M, 10⁻⁸M or 10⁻⁵M leuprolide acetate solution at experiment commencement and on each day after commencement. The number of cells was measured by counting at experiment commencement and on the third and seventh days following commencement, using a hemacytometer and a microscope by a blinded observer. FIG. 1C presents the results of this experiment. For each concentration of leuprolide acetate used in this experiment, and for each day on which cells were counted, FIG. 1C shows, on the vertical axis, the number of cells per plate.

As presented in FIG. 1A-C, with daily administration of the highest concentration (10⁻⁵ M) of leuprolide acetate, growth of DU 145 prostate cancer cells was inhibited by approximately 40% compared to control cells (growing in culture medium which included no leuprolide acetate). With this highest concentration of leuprolide acetate, daily administration resulted in very little growth of DU145 prostate cancer cells between experiment commencement and the seventh day after commencement.

Experiment 2

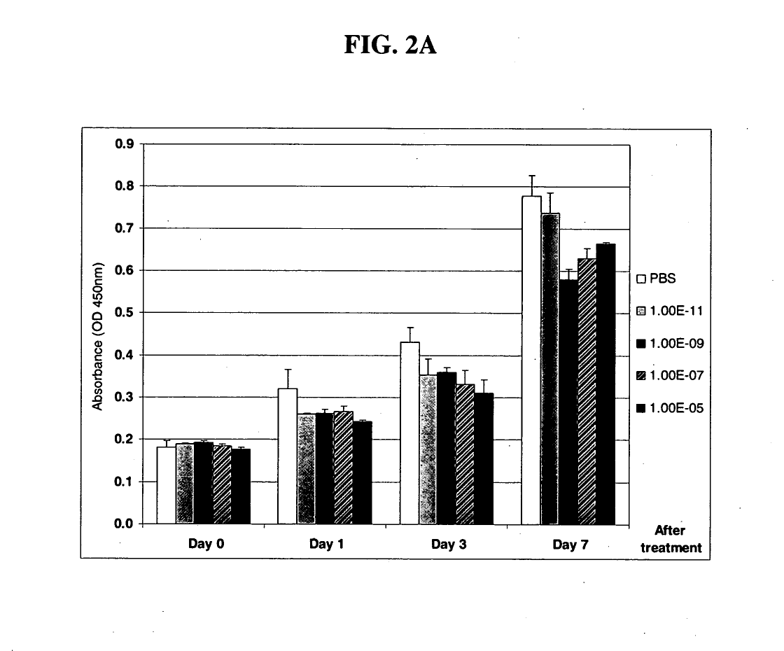

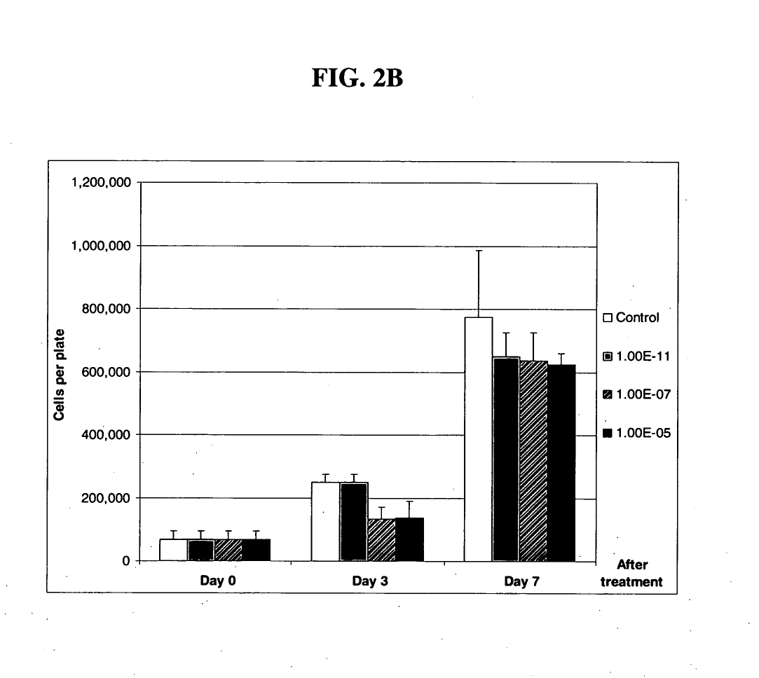

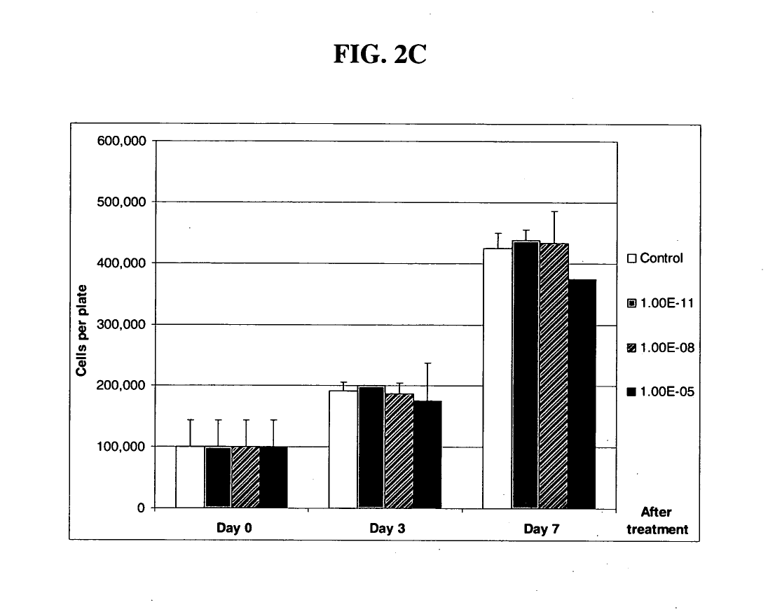

FIGS. 2A-C present results of a series of three experiments on the effects of administration of leuprolide acetate at various molar concentrations on the growth of cells of the PC3 recurrent prostate cancer cell line (ATCC CRL-1435). These cells were from a bone metastasis from a patient with a high grade prostate cancer. Their characteristics include androgen insensitivity for growth and absence of expression of the androgen receptor. These cells were prepared, treated and analyzed using the procedures and techniques described under Experimental Design, above.

FIG. 2A presents the results of the experiment with groups of PC3 cells respectively administered the concentrations of leuprolide acetate identified on FIG. 2A at experiment commencement (0 M, 10⁻¹¹M, 10⁻⁹M, 10⁻⁷M, 10⁻⁵ M).

FIG. 2B presents the results of the experiment with groups of PC3 cells respectively administered the concentrations of leuprolide acetate identified on FIG. 2B at experiment commencement and on the third day after commencement (0 M, 10⁻¹¹M, 10⁻⁷M, 10⁻⁵ M).

FIG. 2C presents the results of the experiment with groups of PC3 cells respectively administered the concentrations of leuprolide acetate identified on FIG. 2C at experiment commencement and on each day after commencement (0 M, 10⁻¹¹ M, 10⁻⁸ M, 10⁻⁵ M).

As presented in FIG. 2A-C, administration of the highest concentration (10⁻⁵ M) of leuprolide acetate inhibited growth of PC3 prostate cancer cells by approximately 20% compared to control cells growing in cell culture medium that included no leuprolide acetate.

Experiment 3

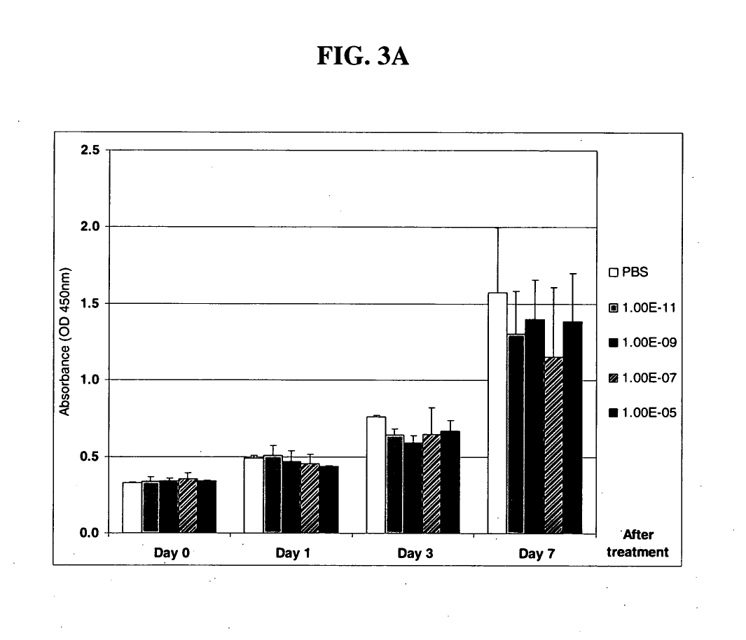

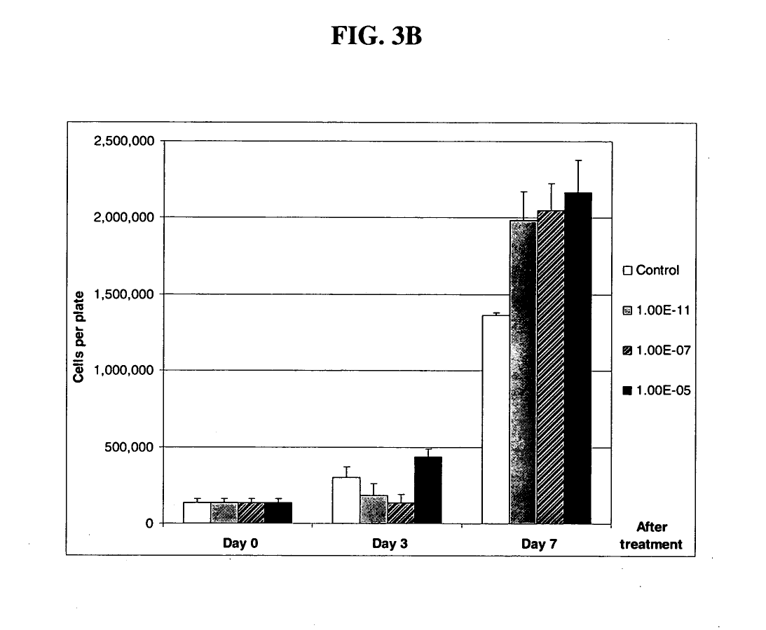

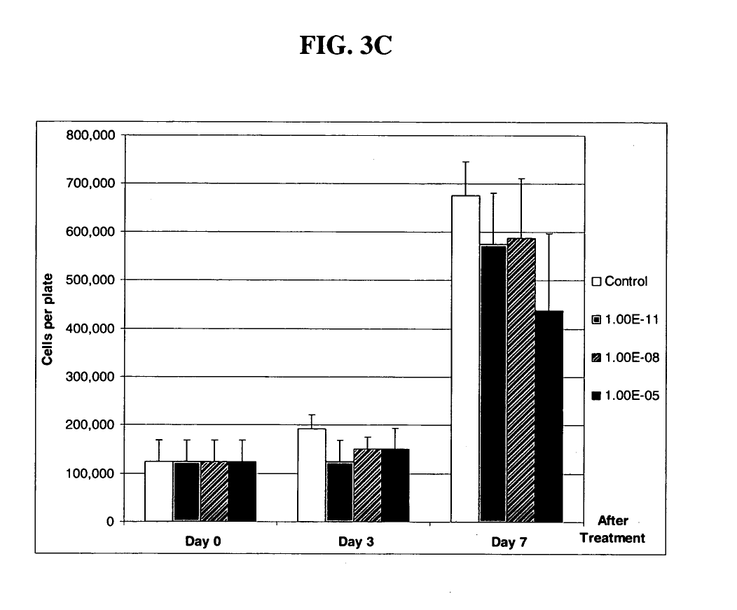

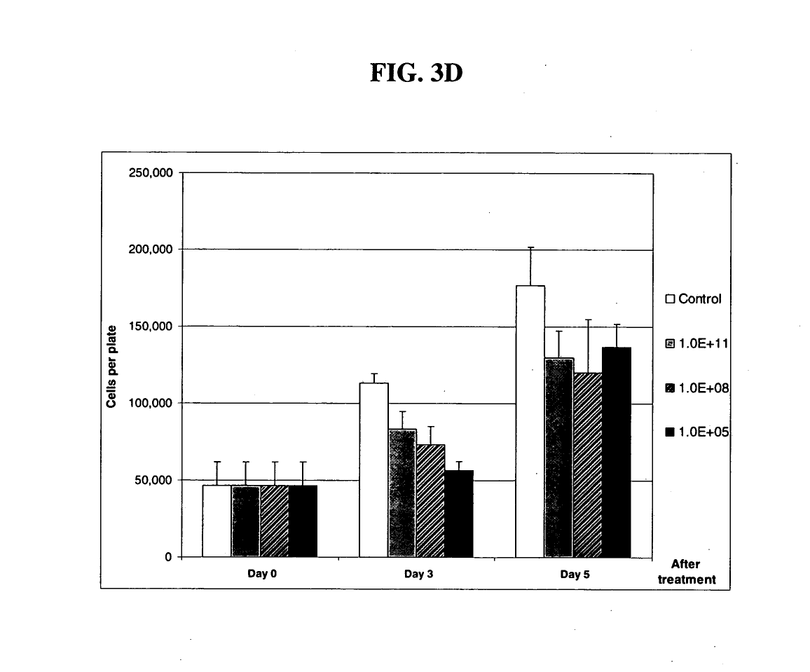

FIGS. 3A-D present results of a series of four experiments on the effects of administration of leuprolide acetate at various molar concentrations on the growth of cells of the CWR-R1 recurrent prostate cancer cell line (described in Gregory C W, Johnson R T Jr., Mohler J L, French F S, Wilson E M. Androgen receptor stabilization in recurrent prostate cancer is associated with hypersensitivity to low androgen. Cancer Res. 61:2892-2898, 2001). These cells were from a recurrent human prostate cancer xenograft initially derived from a patient with hormone-refractory disease. Their characteristics include androgen sensitivity but not dependence for growth and high levels of expression of the androgen receptor. These cells were prepared, treated and analyzed using the procedures and techniques described under Experimental Design, above.

FIG. 3A presents the results of the experiment with groups of CWR-R1 cells respectively administered the concentrations of leuprolide acetate identified on FIG. 3A at experiment commencement (0 M, 10⁻¹¹M, 10⁻⁹M, 10⁻⁷M, 10⁻⁵ M).

FIG. 3B presents the results of the experiment with groups of CWR-R1 cells respectively administered the concentrations of leuprolide acetate identified on FIG. 3B at experiment commencement and on the third day after commencement (0 M, 10⁻¹¹M, 10⁻⁸M, 10⁻⁵ M).

FIG. 3C presents the results of the experiment with groups of CWR-R1 cells respectively administered the concentrations of leuprolide acetate identified on FIG. 3C at experiment commencement and on each day after commencement (0 M, 10⁻¹¹M, 10⁻⁸M, 10⁻⁵ M).

FIG. 3D presents the results of the experiment with groups of CWR-R1 cells respectively administered the concentrations of leuprolide acetate identified on FIG. 3D at experiment commencement and twice daily after commencement for five days (0 M, 10⁻¹¹M, 10⁻⁸ M, 10⁻⁵ M).

As presented in FIG. 3A-D, daily administration of the highest concentration (10⁻⁵M) of leuprolide acetate inhibited growth of CWR-R1 prostate cancer cells by approximately 36% compared to control cells (growing in culture medium which included no leuprolide acetate). As presented in FIG. 3D, twice daily administration of leuprolide acetate inhibited growth of CWR-R1 cells by 35% on day 5 after commencement of treatment compared to cells growing in culture medium which included no leuprolide acetate. The data support that continuous, high dose leuprolide administration inhibits the growth of CWR-R1 recurrent prostate cancer cells.

Experiment 4

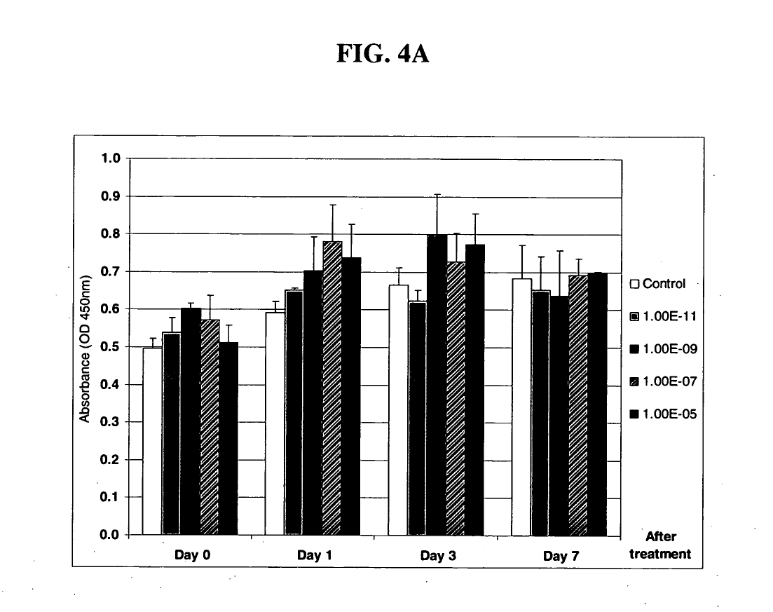

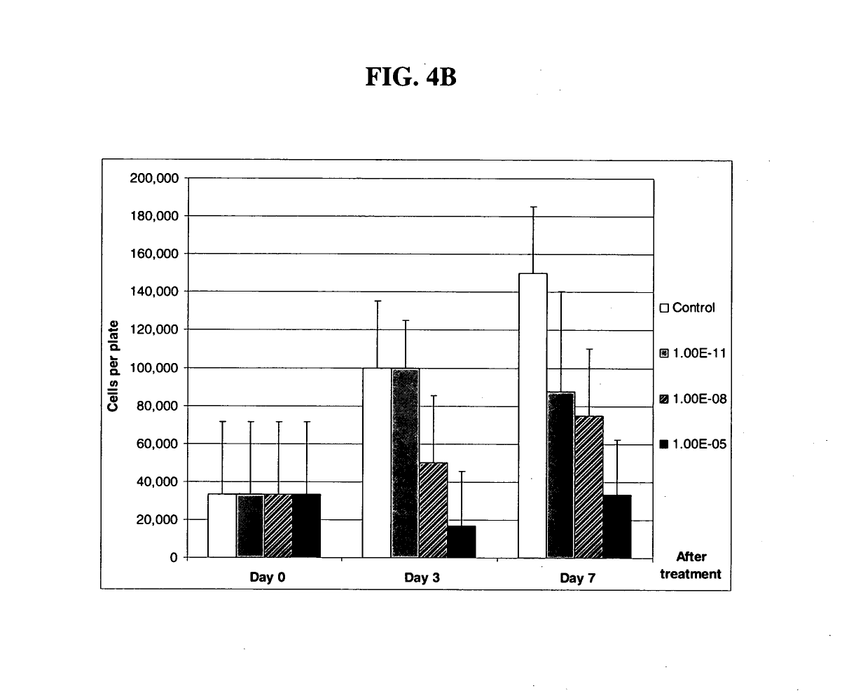

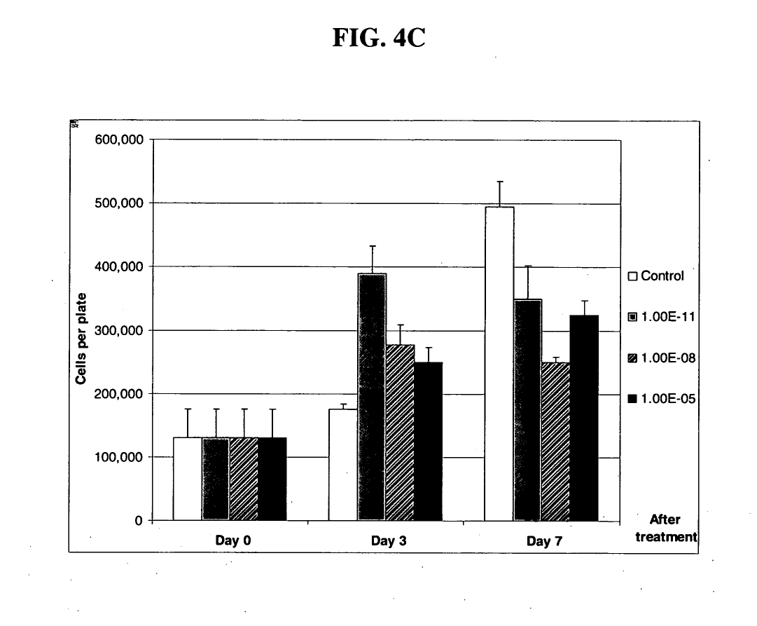

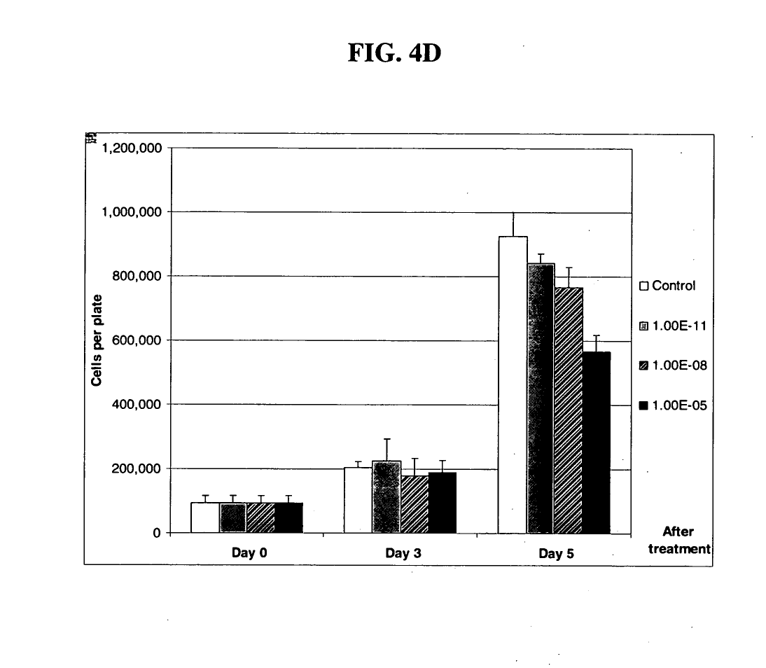

FIGS. 4A-D present results of a series of experiments on the effects of administration of leuprolide acetate at various molar concentrations on the growth of the LNCaP prostate cancer cell line (ATCC CRL-1740). These cells were derived from a supraclavicular lymph node from a man with metastatic prostate cancer. Their characteristics include androgen responsiveness for growth and expression of the androgen receptor. These cells were prepared, treated and analyzed using the procedures and techniques described under Experimental Design, above.

FIG. 4A presents the results of the experiment with groups of LNCaP cells respectively administered the concentrations of leuprolide acetate identified on FIG. 4A at experiment commencement (0 M, 10⁻¹¹M, 10⁻⁹M, 10⁻⁷M, 10⁻⁵ M).

FIGS. 4B and 4C present the results of experiments with groups of LNCaP cells respectively administered the concentrations of leuprolide acetate identified on FIG. 4B and 4C at experiment commencement and on each day after commencement (0 M, 10⁻¹¹M, 10⁻⁸M, 10⁻⁵ M). 20,000 cells were initially plated for the experiment represented in FIG. 4B and 100,000 cells were initially plated for the experiment represented in FIG. 4C.

As presented in FIG. 4B, with daily administration the highest concentration (10⁻⁵M) of leuprolide acetate used in these two experiments, growth in number of LNCaP prostate cancer cells was approximately 70% less than in the number of control cells growing in culture medium which included no leuprolide acetate.

FIG. 4D presents results of an experiment using cells from the LNCaP cell line (ATCC CRL-1740), prepared according to the procedures and techniques described above with respect to Experiment 4, except that (a) cell numbers were counted at experiment commencement and on the third and fifth days following commencement, and (b) the concentrations of leuprolide acetate solution identified on FIG. 4D were respectively administered to each group of cells twice each day from commencement through the five-day experiment period.

As presented in FIGS. 4C and 4D, the administration of the highest leuprolide acetate concentrations (10⁻⁸ M and 10⁻⁵M) once a day and twice a day inhibited growth of LNCaP cells to approximately the same degree. With once daily administration, growth in number of LNCaP prostate cancer cells was approximately 34% less (on the seventh day) compared to cells growing culture medium which included no leuprolide acetate; with twice daily administration, growth in number of LNCaP prostate cancer cells was approximately 39% less (on the fifth day) than the cells growing in culture medium.

Experiment 5

FIGS. 5A and 5B present results of experiments in which 1.5×10⁶ cells of the LNCaP human prostate cancer cell line (ATCC CRL-1740) were injected bilaterally into two groups, each with four mice. One week prior to the injection, a controlled-release leuprolide acetate formulation was implanted into each mouse from one of the groups. Four centimeters of leuprolide rod, providing 22.5 mg of leuprolide was implanted in each mouse of the treatment group. Four centimeters of placebo rod (without leuprolide) was implanted one week prior to injection into each mouse of the other group (the control group).

FIG. 5A presents results of tumor xenograft growth over time in a placebo group and a leuprolide implant group. As FIG. 5A shows, tumor volume measurements were commenced on the twenty-fourth day following injection when tumors were detectable in both groups. By the forty-ninth day following injection, tumors in the control group (n=8) had grown to approximately 540 mm³ on average, while tumors in the treatment group (n=8) had grown to only approximately 240 mm³ on average.

FIG. 5B presents results of measurement of tumor growth rate in each of the treatment and control groups for this experiment. As indicated with respect to FIG. 5B, on the twenty-fourth day following injection, tumors were first observed in both groups and these tumor sizes were used as V₀ for a calculation of growth rate, as described in Experimental Design, above. Tumor growth rates were similar in both groups but tumor volumes were statistically different between groups (FIG. 5A).

Experiment 6

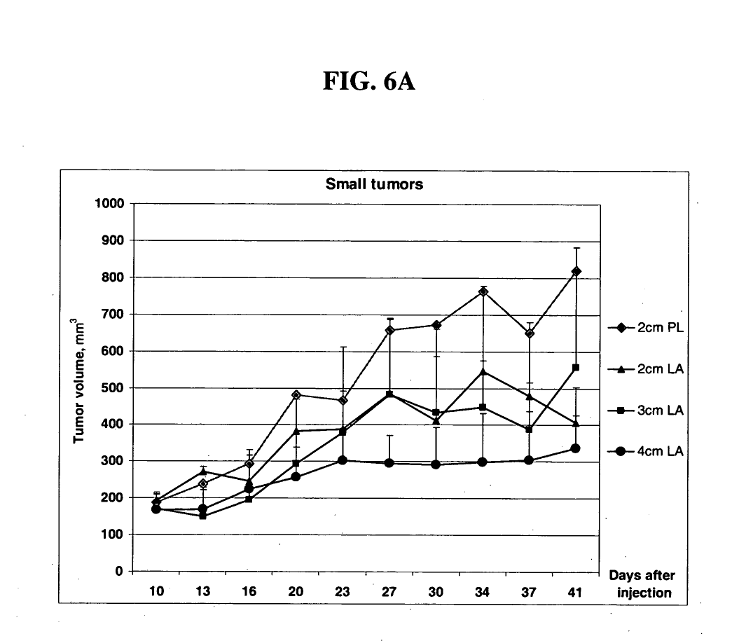

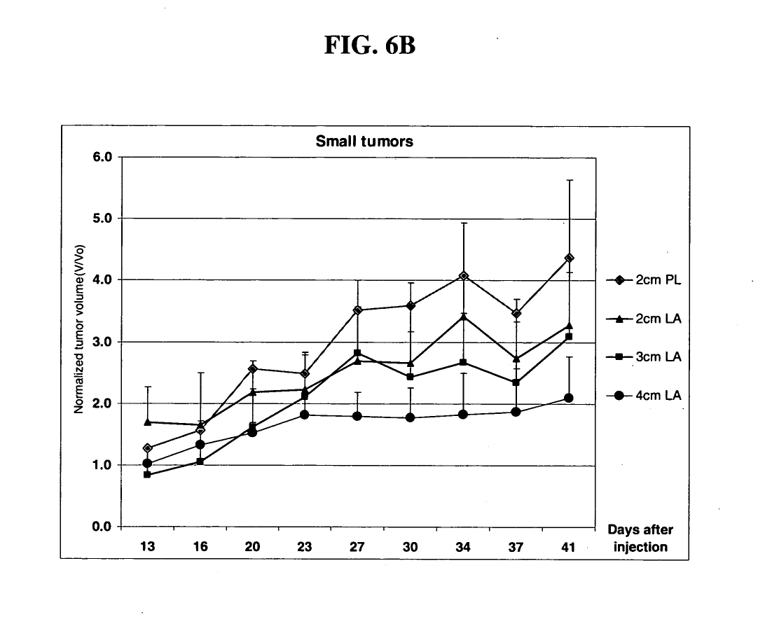

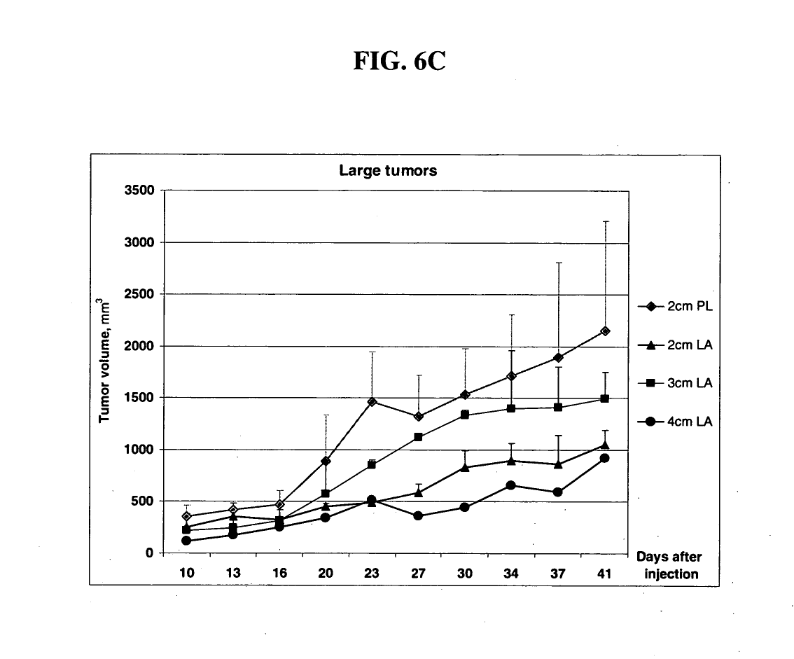

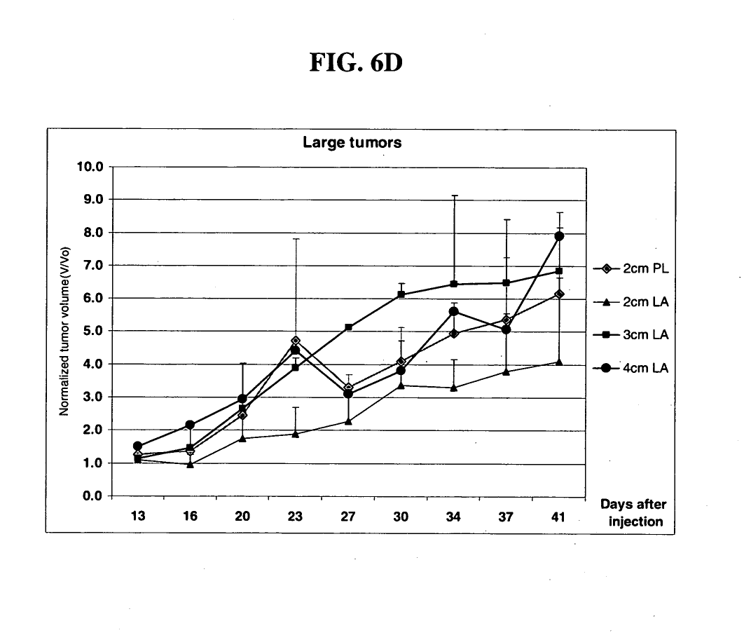

FIGS. 6A-D present results of an experiment in which 5×10⁶ cells from the DU 145 human recurrent prostate cancer cell line were injected into four groups of three mice. Concurrently with this injection, a controlled release formulation, described above, was implanted into the mice from each group, providing the following amounts of leuprolide acetate: placebo (2 cm of formulation, 0 mg leuprolide acetate); low dose (2 cm of formulation, 11.25 mg leuprolide acetate); medium dose (3 cm of formulation, 16.875 mg leuprolide acetate); high dose (4 cm of formulation, 22.5 mg leuprolide acetate). Two subgroups of tumors formed, based on size: large tumors where large is defined as tumors that are ≧4×V₀ and small tumors that are defined as tumors ≦4×V₀. Results are presented by subgroup analysis. Table 1 below shows the percentage of large tumors observed in each group, with the percentage of large tumors in the placebo group higher than in any of the other groups, and more than six times higher than the percentage of large tumors in the high dose leuprolide group.

TABLE 1
(Experiment 6)

| | | Number of | |
|---|---|---|---|
| Treatment Group | Number of Mice | Tumors | % of Large Tumors |
| Placebo | 3 | 5 | 80% |
| Low Dose | 3 | 6 | 33% |
| Medium Dose | 3 | 6 | 50% |
| High Dose | 3 | 6 | 13% |

FIGS. 6A and 6B present results of measurements of the size of small tumors in each group. As FIGS. 6A and 6B show for example, by day 41 following injection, small tumors in the placebo group had grown to more than 800 mm³ on average, while small tumors in the high dose group had grown to approximately 325 mm³ on average. Tumor growth rates for all leuprolide treated groups was slower compared to the placebo group. By day 41, tumors in placebo mice were increased in size four-fold while tumors in the leuprolide treated group were only increased by two-fold.

FIGS. 6C and 6D present results of measurements of the size of large tumors in each group. As FIGS. 6C and 6D show for example, by day 41 following injection, large tumors in the placebo group had grown to more than 2000 mm³ on average, while the single large tumor in the high dose leuprolide group had grown to approximately 900 mm³ on average. While large tumors' growth rates in all groups were similar, tumor sizes were significantly different between placebo and high dose leuprolide groups.

Experiment 7

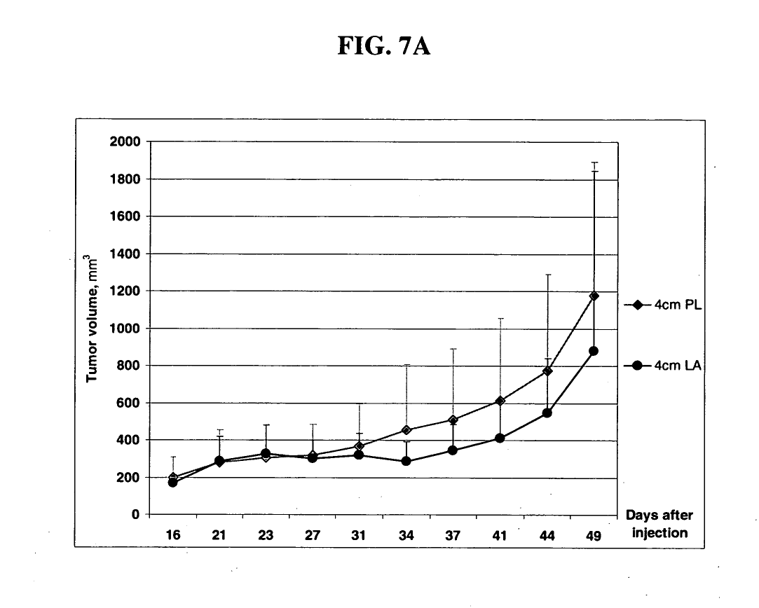

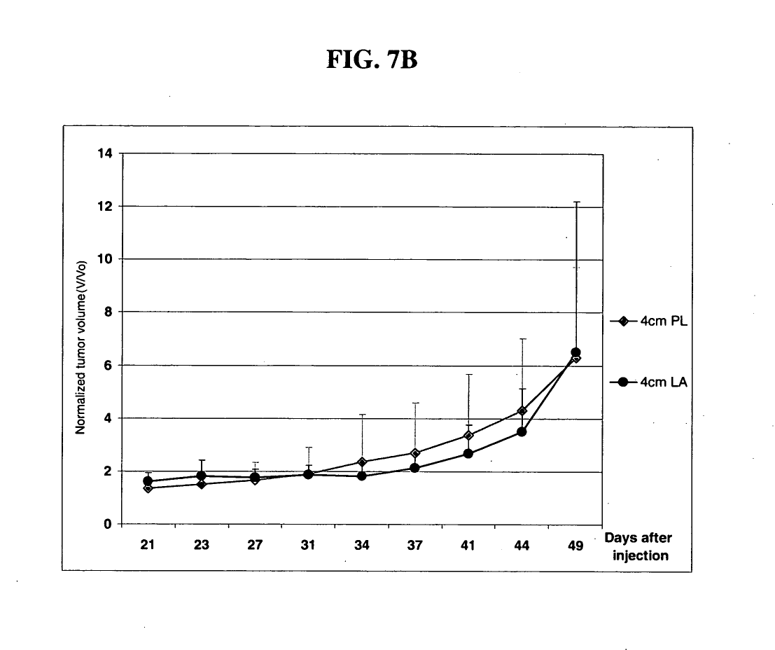

FIGS. 7A and 7B present results of experiments in which 1.5×10⁶ cells of the DU 145 human prostate cancer cell line (ATCC HTB-81) were injected into two groups, each with four mice. One week prior to the injection, a controlled-release leuprolide acetate formulation, described above, was implanted into each mouse from one of the groups. Four centimeters of the formulation, providing 22.5 mg of leuprolide was implanted in each mouse of this treatment group. Four centimeters of placebo rod (without leuprolide) was implanted one week prior to injection into each mouse of the other group (the control group). Eight tumors were formed in each group.

FIG. 7A presents the results of measurements of the size of the tumors in each group. As FIG. 7A shows, by day 49 following injection, tumor size in the control group had increased to approximately 1200 mm³, on average, while tumor size in the treatment group had increased to approximately 900 mm³, on average.

FIG. 7B presents the results of measurement of tumor growth rate for each of the treatment groups in this experiment. As indicated in FIG. 7B, tumors were first observed on day 16 following injection, and the average volume on this day in each group was considered V₀. While tumor growth rates were similar in both groups, the sizes of tumors were 25% smaller in the leuprolide treated group.

Experiment 8

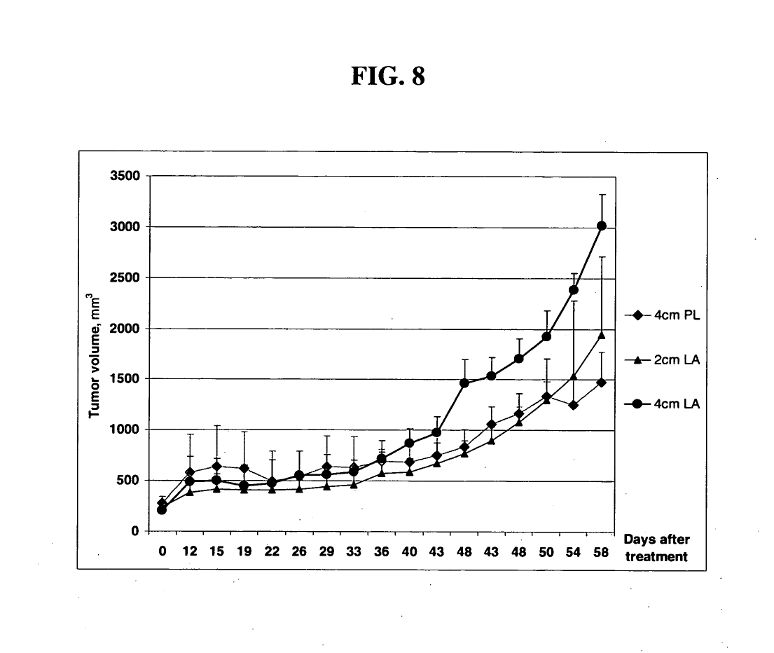

FIG. 8 presents the results of an experiment in which 5×10⁶ cells of the DU145 human prostate cancer cell line (ATCC HTB-81) were injected into two groups, each with three mice. One week after cell injection, placebo (4 cm of formulation without leuprolide) or controlled-release leuprolide implants (2 cm or 4 cm) were inserted into the mice. Tumor volumes were measured over time. While tumor volumes were similar between groups to 43 days after treatment, there was a difference between groups from then out to 58 days after treatment.

Experiment 9

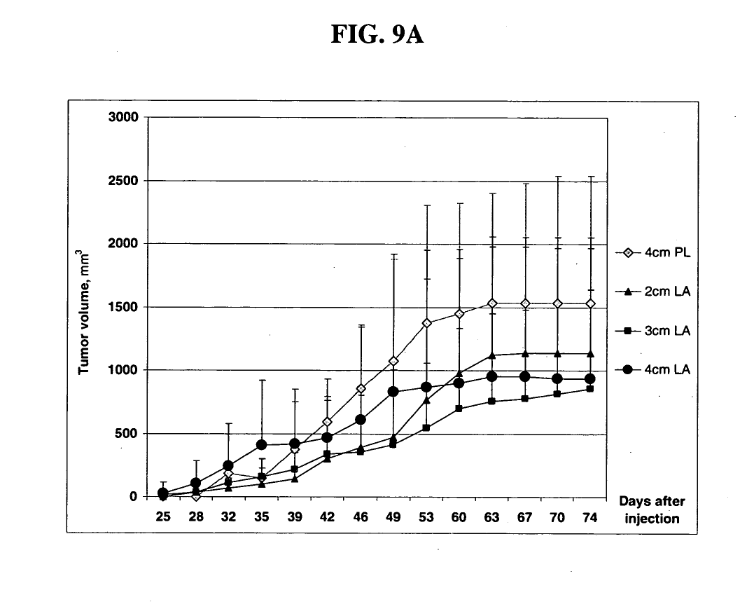

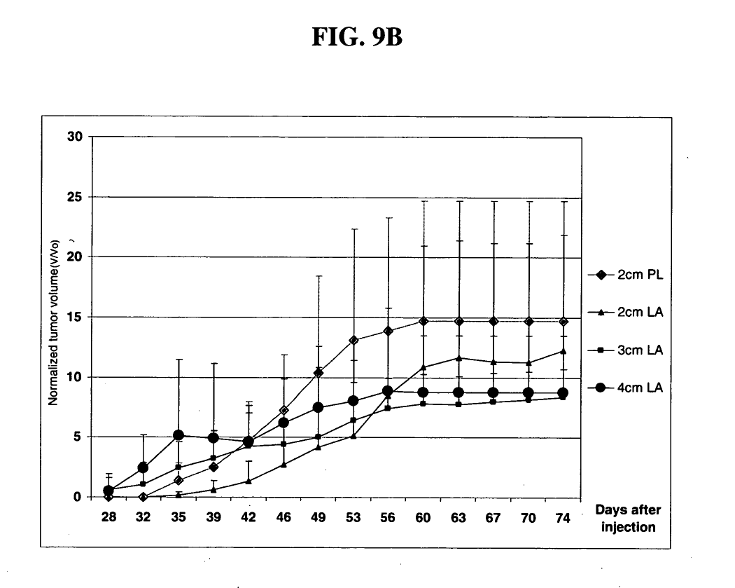

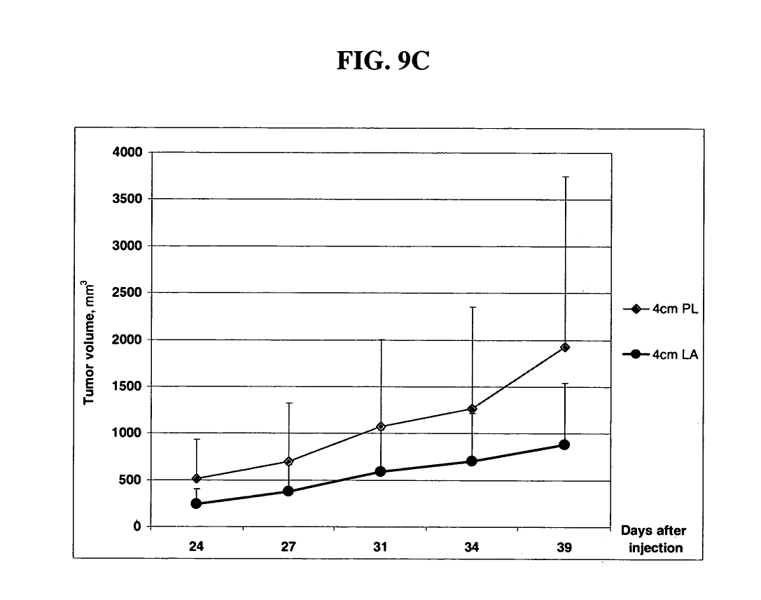

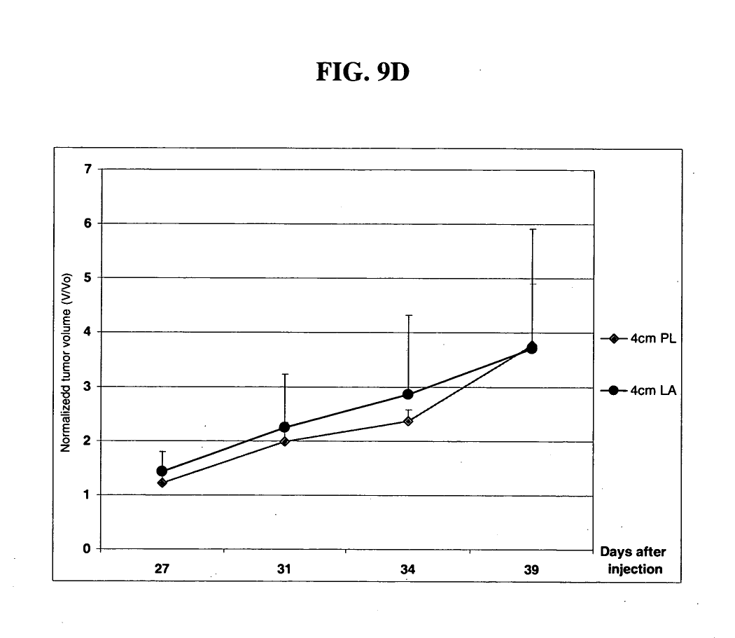

FIGS. 9A and 9B present results of an experiment in which 2×10⁶ cells from the CWR22 human recurrent prostate cancer xenograft (described in Wainstein Mass., He F, Robinson D, Kung H-J, Schwartz S, Giaconia J M, Edgehouse N L, Pretlow T P, Brodner D R, Kursh E D, Resnick M I, Seftel A, Pretlow T G. CWR22: Androgen-dependent xenograft model derived from a primary human prostatic carcinoma. Cancer Res., 54:6049-6052, 1994) were injected into four groups of mice. Concurrently with this injection, a controlled release formulation, described above, was implanted into the mice from each group, providing the following amounts of leuprolide acetate: placebo (2 cm of formulation, 0 mg leuprolide acetate); low dose (2 cm of formulation, 11.25 mg leuprolide acetate); medium dose (3 cm of formulation, 16.875 mg leuprolide acetate); high dose (4 cm of formulation, 22.5 mg leuprolide acetate). Table 2 below shows the number tumors observed in each group.

TABLE 2
(Experiment 9)

| Treatment Group | Number of Mice | Number of Tumors |
|---|---|---|
| Placebo | 3 | 6 |
| Low Dose | 4 | 7 |
| Medium Dose | 4 | 6 |
| High Dose | 4 | 6 |

FIG. 9A presents results of measurements of the size of tumors in each group. As FIG. 9A shows for example, by day 74 following injection, tumors in the placebo group had grown to more than 1500 mm³ on average, while small tumors in the high dose group had grown to approximately 900 mm³ on average.

FIG. 9B presents the results of measurement of small tumor growth rate for each of the four treatment groups in this experiment. As indicated in FIG. 9B, tumors were first observed on day 28 following injection and these average tumor volumes per group were used as V₀. By day 74 following injection, tumor size in the placebo group had increased to a volume approximately fifteen times greater than the average tumor volume first observed in the high dose group, while the tumor size in the high dose group had increased approximately eight times in volume.

FIGS. 9C and 9D present results from an experiment in which 1.4×10⁶ cells from the human CWR22 recurrent prostate cancer xenograft were injected into two groups of mice with four mice each. Concurrent with the injection, mice were implanted with 4 cm of formulation (implants without leuprolide) or 4 cm of leuprolide implants. Results in FIGS. 9C and 9D are similar to the data presented in FIGS. 9A and 9B demonstrating smaller tumors and slower tumor growth in leuprolide-treated mice compared to placebo-mice.

Experiment 10

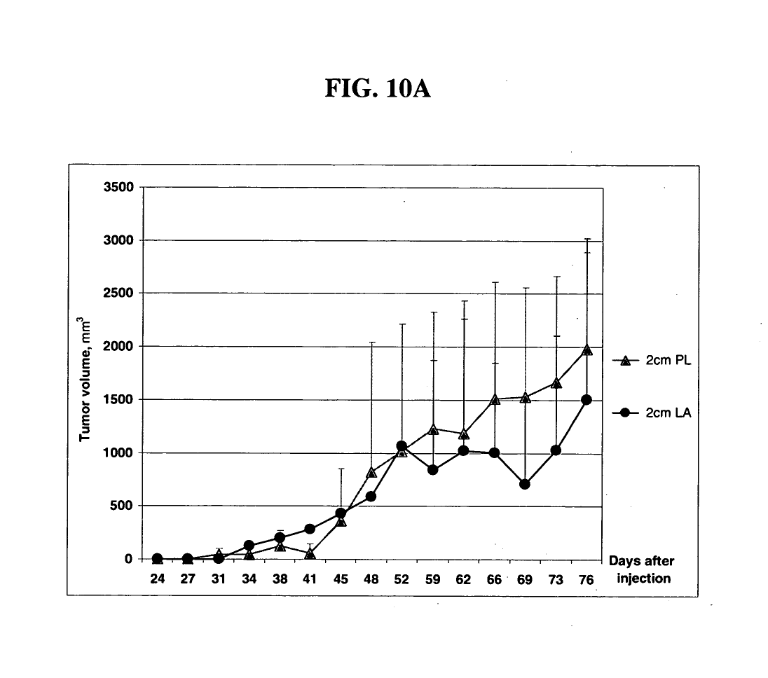

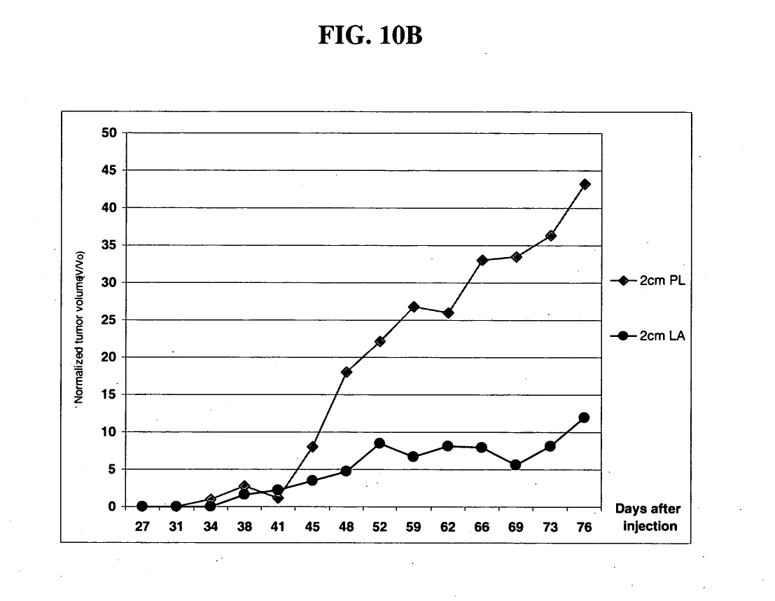

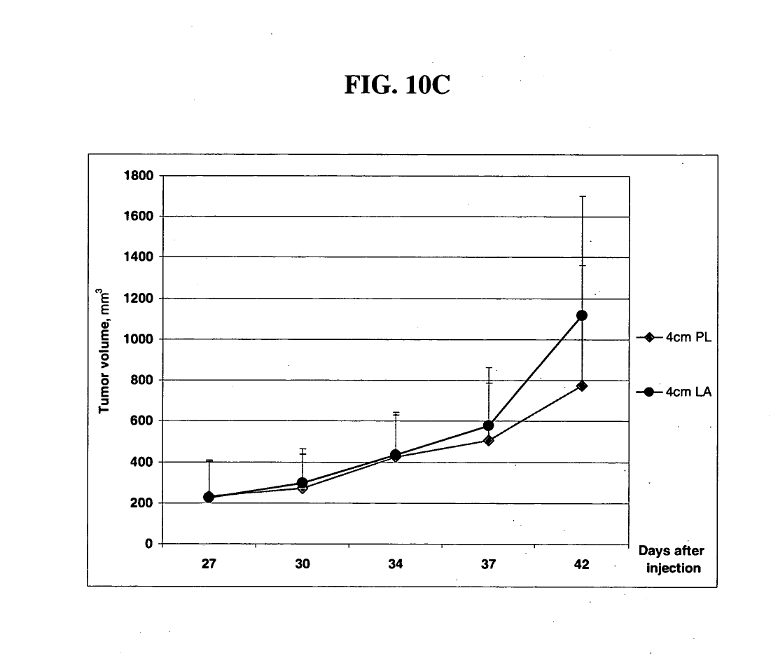

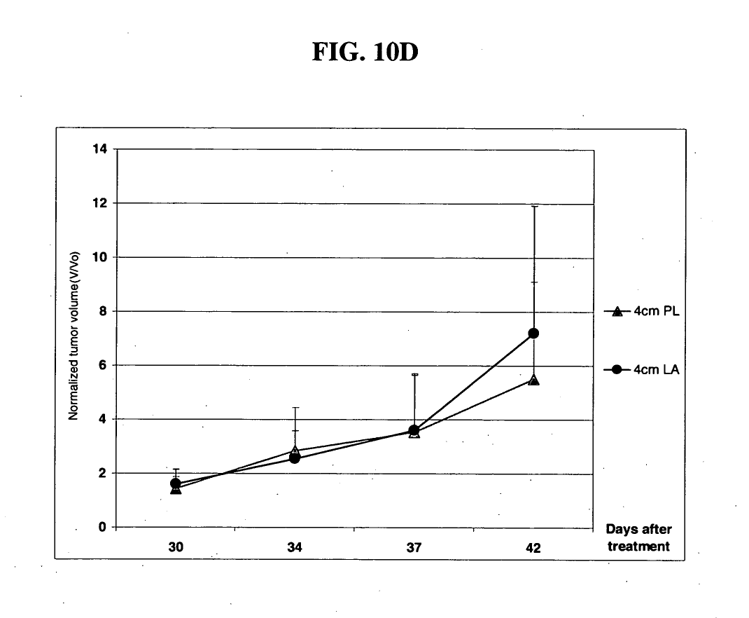

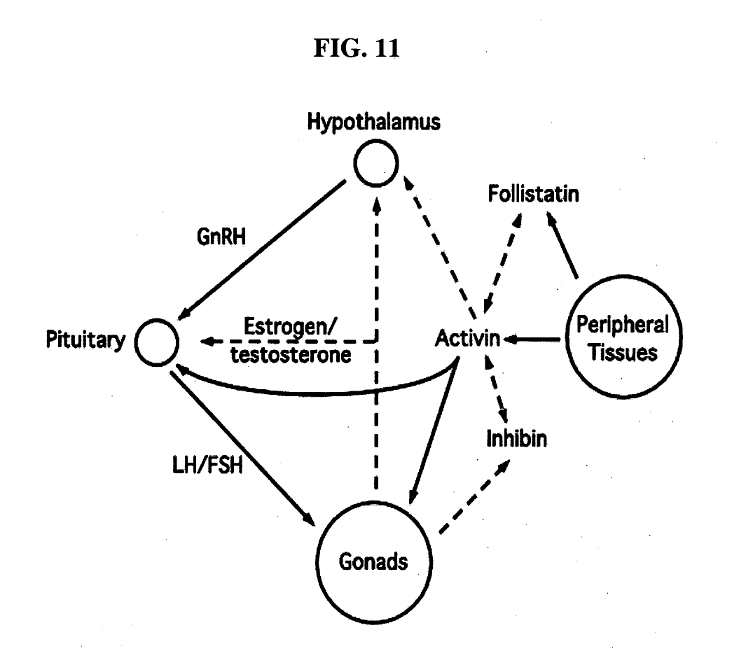

FIGS. 10A and 10B present results of an experiment in which 2×10⁶ cells of the CWR 22 human prostate cancer xenograft were injected into two groups of mice. Four days prior to the injection, a controlled-release leuprolide acetate formulation, described above, was implanted into each of the three mice in the treatment group. Two centimeters of the formulation, providing 11.25 mg of leuprolide was implanted in each mouse of this treatment group. Two centimeters without any leuprolide was implanted four days prior to injection into each of the four mice of the other group (the control group).

In the leuprolide treatment group, tumor formation was first observed on average 54.3 days after injection, while in the control group, tumor formation was first observed on average 27.5 days after injection. Moreover, at day 34 following injection, one tumor was observed in the treatment group (out of three), while four tumors were observed in the control group (out of four).

FIG. 10A presents tumor volume measurements for this experiment. Average tumor volumes in the placebo group on day 76 after injection are 2000 mm³ while average tumor volumes in the leuprolide treated group are 1500 mm³.

FIG. 10B presents tumor growth rate results for this experiment. As FIG. 10B shows, on day 76 after injection, tumor size in the placebo group had increased to more than 40 times the initial volume, which was about 3.5 times the growth rate of tumors in the treatment group.

FIG. 10C presents tumor volume measurements for an experiment using the same protocol, with CWR22 recurrent prostate cancer xenograft cells. Average tumor volumes in the placebo group on day 42 after injection are 800 mm³ while average tumor volumes in the leuprolide treated group are 1100 mm³.

FIG. 10D presents tumor growth rate results for the experiment of FIG. 10C. As FIG. 10D shows, on day 42 after injection, tumor growth rates for both groups are similar.

### Exemplary Embodiments

In an embodiment of this invention, prostate cancer is prevented, delayed, mitigated, or treated by administering a dosage regimen of GnRH agonists or antagonists that is at least about two to three times higher, and in embodiments more than three times higher, than is currently approved for the same indication. Since no toxic dose of GnRH agonists is believed to have been documented, another embodiment of this invention includes treating, preventing, slowing the progression of, or mitigating prostate cancer by continually increasing the dose of the GnRH agonist or antagonist until a decrease in prostate-specific antigen (PSA) is achieved or until the patient develops adverse effects that represent greater risk or discomfort than does the risk or discomfort of the prostate cancer.

In another embodiment of the invention, prostate cancer would be prevented, treated, delayed, or mitigated by directly and constantly infusing GnRH agonists or antagonists into the affected tissue, for example from a reservoir into the prostate via a catheter (such as a fenestrated catheter) embedded directly into the prostate. The drug is thus directly delivered to the prostate rather than indirectly delivered through the bloodstream. It is well known in the art to deliver drugs by infusion through a catheter embedded directly in a part of a patient's body requiring treatment, for example, in the liver of a patient requiring chemotherapy drugs for the treatment of liver cancer.

In other embodiments of the invention, controlled release formulations of GNRH agonists or antagonists would be implanted directly into or near the prostate tissue in order to prevent, treat, delay, or mitigate prostate cancer, for example by injection directly into the prostate using a fine needle in a fashion similar to the way radioisotope seeds are implanted in brachytherapy. This would allow for high prostatic concentrations of the GNRH agonist or antagonist while minimizing peripheral exposure. Currently, in the course of an in vitro fertilization process, a needle may be used to inject about 1 mg/day of GnRH agonists or antagonists into a patient. According to an embodiment of the present invention, a dose of a GnRH agonist or antagonist administered for the treatment, mitigation, delay, or prevention of prostate cancer, when delivered by fine needle injection of controlled release formulations directly into or near the prostate, results in serum and/or prostate tissue levels of at least about 3 ng/ml or more. In embodiments, this level of serum and/or prostate tissue levels of the GnRH agonist or antagonist would be maintained for a period of at least one month, or at least three months or more. In embodiments, the controlled release formulation would be formulated to expose prostate cancer cells of the patient to concentrations of the GnRH agonist or GnRH antagonist that would result from blood serum concentrations of the GnRH agonist or GnRH antagonist of at least about 3 ng/ml for a period of at least one month, or at least three months or more.

In other embodiments of the present invention, the dosage regime of GnRH agonist or antagonist to treat, prevent, mitigate or slow the progression of prostate cancer would be a physiologically equivalent dose to a dose of leuprolide in the range of 11.25 mg/month to 22.5 mg/month, or a dose of an agent resulting in daily dosages physiologically equivalent to a dose of leuprolide of approximately 0.375 mg/day to approximately 0.75 mg/day. In embodiments, the controlled release formulation would be formulated to maintain the tissue concentration of the GnRH agonist or antagonist at levels that produce the same or similar physiological-effects as dosages of leuprolide of 7.5 mg/month, 11.25 mg/month, 22.5 mg/month, or more. In embodiments, the higher tissue concentration would be substantially sustained at a high level instead of spiking initially and briefly to a very high level and then dropping substantially.

In other embodiments of the invention, implanted controlled release formulations of GnRH agonists or antagonists would achieve a release profile that provides a substantially stable serum concentration of GNRH agonists or antagonists that is at least about two to five times the serum concentration (or more, for aggressive cancers) provided by currently-known treatments of prostate cancer using GnRH agonists or antagonists, in which the serum concentration is substantially sustained at the higher level instead of spiking initially and briefly to a very high level and then dropping substantially. For example, an implanted controlled release formulation of the present invention for treating, delaying, preventing or treating prostate cancer would provide a GNRH agonist or antagonist serum concentration of at least about 3 ng/ml, in embodiments up to 10 ng/ml (or more, especially for aggressive cancers), over the lifetime of the formulation. Such formulations, using polymeric controlled release technology, are available from Durect Corporation, Cupertino, Calif.

Other known methods of delivery are also suitable for administering GnRH agonists or antagonists according to the present invention, such as intramuscular injection of microspheres.

Examples of GnRH agonists or antagonists include but are not limited to Antide® brand of iturelix; Lupron® brand of leuprolide acetate; Zoladex® brand of goserelin acetate; Synarel® brand of nafarelin acetate; Trelstar Depot brand of triptorelin; Supprelin brand of histrelin; Suprefact brand of buserelin; Cetrotide® brand of cetrorelix; Plenaxis® brand of abarelix; Antagon brand of ganirelix; and degarelix (FE200486).

Embodiments of the present invention also include treatment, mitigation, slowing the progression of, or preventing prostate cancer by co-administering a GnRH agonist or antagonist with androgen synthesis blockers (5a-reductase inhibitors) or analogues thereof, which include but are not limited to Proscar® brand of finasteride and Avodart® brand of dutasteride.

Further embodiments of the present invention also include co-administration of a GnRH agonist or antagonist with follicle stimulating hormone receptor blockers or analogues thereof which include but are not limited to anti-FSH receptor immunoglobulins.

FSH is currently understood as a cause of prostate cancer, and testosterone can cause a decrease in the production of FSH. In particular contrast to conventional teaching, the present invention includes still further embodiments for treating, mitigating, slowing the progression of, or preventing prostate cancer in which a GnRH agonist or antagonist is co-administered with testosterone or analogues thereof, in order to substantially reduce if not completely shut down production of FSH.

Embodiments of the present invention also include treating, mitigating, slowing the progression of, or preventing prostate cancer by co-administering a GnRH agonist or antagonist with luteinizing hormone receptor blockers or analogues thereof, which include but are not limited to interleukin-1 and anti-LH receptor immunoglobulins; co-administering a GNRH agonist or antagonist with activin receptor blockers or analogues thereof; and administering other agents, including agents not yet known, that decrease the degradation of, or increase the half-life of, or increase prostate tissue levels of GnRH agonists or antagonists.

Additionally, the present invention encompasses pharmaceutical formulations containing GnRH agonists and/or GnRH antagonists and which are configured to be implanted in prostate tissue and to provide serum concentrations or certain tissue concentrations of the GnRH agonists and/or GnRH antagonists substantially higher than serum levels resulting from conventional prostate cancer treatments using GnRH agonists or antagonists. The pharmaceutical formulations could be used, for example, to treat, slow, mitigate, or prevent prostate cancer.

While various embodiments of the present invention have been described throughout this specification, it should be understood that they have been presented by way of example only, and not by way of limitation. For example, the present invention is not limited to the agents illustrated or described. As such, the breadth and scope of the present invention should not be limited to any of the above-described exemplary embodiments, but should be defined in accordance with the appended claims and their equivalents.

What is claimed is:

1. A method for treating hormone refractory prostate cancer in a patient having hormone. refractory prostate cancer, for preventing hormone refractory prostate cancer in a patient at risk of contracting hormone refractory prostate cancer, for decreasing the level of prostate-specific antigen in a patient with hormone refractory prostate cancer, or for preventing or slowing the proliferation of cells of prostate origin in a patient with hormone refractory prostate cancer, comprising:
administering to the patient a therapeutically effective amount of at least one physiological agent that decreases or regulates blood or tissue levels, expression, production, function, or activity of at least one of luteinizing hormone (LH), LH receptors, follicle stimulating hormone (FSH), FSH receptors, an androgenic steroid, androgenic steroid receptors, an activin, and activin receptors,
wherein the physiological agent is a GnRH agonist or antagonist wherein the dose used is effective to achieve a blood serum level of at least 3 ng/ml of the physiological agent for at least one month in the patient.

2. A method for treating hormone refractory prostate cancer in a patient having hormone refractory prostate cancer, for preventing hormone refractory prostate cancer in a patient at risk of contracting hormone refractory prostate cancer, for decreasing the level of prostate-specific antigen in a patient with hormone refractory prostate cancer, or for preventing or slowing proliferation of cells of prostate origin in a patient with hormone refractory prostate cancer, comprising:
administering to the patient a therapeutically effective amount of at least one physiological agent that increases or regulates blood or tissue levels, expression, production, function, or activity of at least one of gonadotropin releasing hormone (GnRH), an inhibin, and a follistatin,
wherein the physiological agent is a GnRH agonist or antagonist wherein the dose used is effective to achieve a blood serum level of at least 3 ng/ml of the physiological agent for at least one month in the patient.

3. A method of preventing or inhibiting an upregulation of the cell cycle in prostate-derived hormone refractory prostate cancer cells in a patient, comprising:
administering to the patient an amount of at least one physiological agent selected from the group consisting of GnRH agonists and GnRH antagonists, effective to reduce local tissue production of hormones of the hypothalamic-pituitary-gonadal (HPG) axis.

4. A method of treating hormone refractory prostate cancer in a patient having hormone refractory prostate cancer, comprising:
administering to the patient an amount of at least one physiological agent selected from the group consisting of GnRH agonists and GnRH antagonists, effective to achieve a blood serum level of at least 3 ng/ml of the physiological agent for a predetermined time interval.

5. A method for treating hormone refractory prostate cancer in a patient having hormone refractory prostate cancer, comprising:
administering to the patient an initial dose of a GnRH agonist or a GnRH antagonist; wherein the dose used is effective to achieve a blood serum level of at least 3 ng/ml of the physiological agent for at least one month in the patient; and
monitoring for decreases in prostate-specific antigen level in the patient, and subsequently administering to the patient increasing doses of the GNRH agonist or the GnRH antagonist until no further decrease in prostate-specific antigen level in the patient is observed.

6. A method for treating hormone refractory prostate cancer in a patient having hormone refractory prostate cancer, comprising:
administering to the patient a therapeutically effective amount at least one physiological agent selected from the group consisting of GnRH agonists and GNRH antagonists by substantially continuously infusing the physiological agent directly into the prostate of the patient so that hormone refractory prostate cancer cells are exposed to concentrations of the physiological agent of at least 3 ng/ml.

7. The method of claim 1, wherein the at least one physiological agent is one of: gonadotropin releasing hormone (GnRH); a GnRH agonist selected from the group consisting of leuprolide, goserelin, nafarelin, triptorelin, histrelin, and buserelin; a GnRH antagonist selected from the group consisting of iturelix, cetrorelix, abarelix, ganirelix, and degarelix; an inhibin; beta-glycan; and a follistatin.

8. The method of any one of claims 1-3, wherein the at least one physiological agent is leuprolide, and the therapeutically effective amount is in the range of approximately 11.25 mg/month to at least approximately 22.5 mg/month.

9. The method of any one of claims 1-3, wherein the therapeutically effective amount of the at least one physiological agent is an amount of the physiological agent, administered or released over a predetermined time period, targeted to achieve substantially equivalent physiological effects as those resulting from a blood serum level of leuprolide of at least about 3 ng/ml of leuprolide over about the predetermined time period wherein the time period is selected from: at least 1 month; and at least 3 months .

10. A method for treating hormone refractory prostate cancer in a patient having hormone refractory prostate cancer, comprising:
administering to the patient a therapeutically effective amount of at least one physiological agent selected from the group consisting of GnRH agonists and GnRH antagonists, by implanting a pharmaceutical controlled release formulation of the at least one physiological agent directly into or near the prostate tissue of the patient.

11. The method of claim 10, wherein the pharmaceutical controlled release formulation is formulated to provide a serum concentration of the at least one physiological agent of at least about 3 ng/ml maintained for a period of at least about one month.

12. The method of claim 10, wherein the pharmaceutical controlled release formulation is formulated to expose hormone refractory prostate cancer cells of the patient to concentrations of the at least one physiological agent resulting from a blood serum concentration of the at least one physiological agent of at least about 3 ng/ml for a period of at least about one month.

13. A method for treating hormone refractory prostate cancer in a patient having hormone refractory prostate cancer, comprising:
administering to the patient a first physiological agent selected from the group consisting of GnRH agonists and GnRH antagonists in a therapeutically effective combination with a second physiological agent selected from the group consisting of androgen synthesis blockers, analogues of androgen synthesis blockers, FSH receptor blockers, analogues of FSH receptor blockers, testosterone, testosterone analogues, LH receptor blockers, analogues of LH receptor blockers, activin blockers, and analogues of activin blockers.

14. A method for treating hormone refractory prostate cancer in a patient having hormone refractory prostate cancer, comprising:
administering to the patient having hormone refractory prostate cancer a physiological agent that decreases the degradation of GnRH agonists or GnRH antagonists within the patient, increases the half-life of GnRH agonists or GnRH antagonists within the patient, or increases prostate tissue levels of GnRH agonists or GnRH antagonists within the patient.
